(19) United States
(12) Patent Application Publication
(10) Pub. No.: US 2021/0113501 A1
(43) Pub. Date: Apr. 22, 2021

(54) METHOD OF ADMINISTERING BETA-HYDROXY-BETA-METHYLBUTYRATE (HMB)

(71) Applicant: Metabolic Technologies, Inc., Ames, IA (US)

(72) Inventors: John Rathmacher, Story City, IA (US); John Fuller, JR., Zearing, IA (US); Shawn Baier, Polk City, IA (US); Steve Nissen, Ames, IA (US); Naji Abumrad, Nashville, TN (US)

(21) Appl. No.: 17/076,493

(22) Filed: Oct. 21, 2020

Related U.S. Application Data
(63) Continuation-in-part of application No. 12/973,803, filed on Dec. 20, 2010, now abandoned.
(60) Provisional application No. 61/287,857, filed on Dec. 18, 2009.

(51) Int. Cl. A61K 31/19; A23L 29/00; A23L 33/00

(52) U.S. Cl. CPC A61K 31/19; A23L 33/30; A23L 29/03

(57) ABSTRACT

A method of administering beta-hydroxy-beta-methylbutyric acid (HMB) is described, and specifically administering HMB-acid to a person such that the administration of free acid HMB results in an increase in effectiveness of HMB over administration of other forms of HMB, including a calcium salt HMB composition is described. Administration of HMB-acid results in an increase in the peak level of HMB in plasma compared with administration of a similar dose of a calcium salt HMB composition. Administration of HMB-acid results in a faster time to reach peak plasma levels of HMB relative to administration of a similar dosage of a calcium salt HMB composition. Administration of HMB-acid results in improved bioavailability and improved pharmokinetic parameters compared to administration of the calcium salt form of HMB.

This application is a continuation-in-part of U.S. patent application Ser. No. 12/973,803 filed Dec. 20, 2010, which claims priority to U.S. Patent Application Ser. No. 61/287,857 filed Dec. 18, 2009, which is herein incorporated by reference in its entirety.

### FIELD OF THE INVENTION

The invention relates generally to a more efficient and more effective delivery system for β-Hydroxy-β-methylbutyrate (HMB) and more specifically to administration of HMB free acid resulting in a more efficient and more effective way of administrating HMB over administration of a similar dosage of the calcium salt form of HMB (CaHMB).

### BACKGROUND OF THE INVENTION

β-Hydroxy-β-methylbutyrate can also be designated by its IUPAC name 3-hydroxy-3-methylbutyric acid or β-hydroxy-β-methylbutyric acid. The abbreviation “HMB” is used to describe β-Hydroxy-β-methylbutyrate and/or 3-hydroxy-3-methylbutyric acid and/or β-hydroxy-3-methylbutyric acid. The acronym “HMB” has been synonymous with the calcium salt form of β-Hydroxy-β-methylbutyrate (also known as 3-hydroxy-3-methylbutyric acid) which has been the commercially produced form for more than twenty-five years. The acronym “HMB” is used in the titles of studies using calcium HMB (CaHMB), including those in this Table A:

TABLE A

| Title | Reference |
|---|---|
| Effect of feeding Beta-hydroxy-Beta- | (Moschini, Rathmacher et al. 1993) |
| methylbutyrate (HMB) on leucine and fat | |
| metabolism in mammary gland | |
| The effects of the leucine catabolite β-hydroxy-β- | (Nissen, Morrical et al. 1994) |
| methylbutyrate (HMB) on the growth and health of | |
| growing lambs | |
| Effect of β-hydroxy-β-methyl butyrate (HMB) | (Nissen, Panton et al. 1996) |
| supplementation on strength and body composition | |
| of trained and untrained males undergoing intense | |
| resistance training | |
| Effect of supplemental β-hydroxy β-methylbutyrate | (Nissen, Clark et al. 1999) |
| (HMB), glutamine, and arginine on skeletal muscle | |
| mass in AIDS patients | |
| Effects of calcium beta-hydroxy-beta- | (Kreider, Ferreira et al. 1999) |
| methylbutyrate (HMB) supplementation during | |
| resistance-training on markers of catabolism, body | |
| composition and strength | |
| Effects of combined beta-hydroxy-beta- | (Shirato, Tsuchiya et al. 2016) |
| methylbutyrate (HMB) and whey protein ingestion | |
| on symptoms of eccentric exercise-induced muscle | |
| damage | |
| Effects of beta-hydroxy-beta-methylbutyrate (HMB) | (Standley, Distefano et al. 2017) |
| on skeletal muscle mitochondrial content and | |
| dynamics, and lipids after 10 days of bed rest in | |
| older adults | |
| The effect of a 12-week beta-hydroxy-beta- | (Durkalec-Michalski, Jeszka et al. 2017) |
| methylbutyrate (HMB) supplementation on highly- | |
| trained combat sports athletes: A randomised, | |
| double-blind, placebo-controlled crossover study | |

In the absence of additional information to designate the form of HMB, the following terms are used in the literature to designate CaHMB:
β-Hydroxy-β-methylbutyrate
β-hydroxy-β-methylbutyric acid
3-hydroxy-3-methylbutyric acid
HMB
CaHMB
Calcium HMB
HMB in the calcium salt form

β-hydroxy-β-methylbutyric acid or β-Hydroxy-β-methylbutyrate can be used in the free acid form (HMB-acid) or as an edible salt, typically an edible calcium salt (CaHMB).

HMB has been found to be useful within the context of a variety of applications. Specifically, in U.S. Pat. No. 5,360,613 (Nissen), HMB is described as useful for reducing blood levels of total cholesterol and low-density lipoprotein cholesterol. In U.S. Pat. No. 5,348,979 (Nissen et al.), HMB is described as useful for promoting nitrogen retention in humans. U.S. Pat. No. 5,028,440 (Nissen) discusses the usefulness of HMB to increase lean tissue development in animals. Also, in U.S. Pat. No. 4,992,470 (Nissen), HMB is described as effective in enhancing the immune response of mammals. U.S. Pat. No. 6,031,000 (Nissen et al.) describes use of HMB and at least one amino acid to treat disease-associated wasting. In U.S. Pat. No. 6,103,764, HMB is described as increasing the aerobic capacity of muscle of an animal without a substantial increase in the mass of the muscle. In addition, HMB has been described as useful for improving a human's perception of his emotional state in U.S. Pat. No. 6,291,525.

HMB has been shown to have positive effects on maintaining and increasing lean muscle mass in cancer cachexia and AIDS wasting. In addition, a positive effect on muscle damage and the resulting inflammatory response caused by exercising which leads to muscle soreness, strength loss, and an increase in pro-inflammatory cytokines is seen with use of HMB.

It has previously been observed that HMB alone or in combination with other amino acids is an effective supplement for restoring muscle strength and function in young athletes. Further, it has been observed that HMB in combination with two amino acids, arginine and lysine, is effective in increasing muscle mass in elderly persons.

Approaches for improving muscle function, including increasing muscle strength, improving muscle performance, and increasing muscle mass include using nutritional and/or dietary supplements. Use of nutraceuticals and/or dietary supplements is popular with athletes and/or bodybuilders as a way to enhance muscle function and performance. Other populations in need of improvements in muscle function, mass and performance include anyone experiencing or at risk of experiencing loss of muscle strength, function, performance and/or mass such as the elderly, people recovering from surgery, people undergoing long periods of inactivity, people suffering from muscle wasting, cancer patients, etc. As athletes, or any person desiring to improve muscle health, continually strive for improved muscle function, strength, and/or performance and/or to increase mass, there is a continuing need for new, more effective technologies to achieve these goals.

HMB is an active metabolite of the amino acid leucine. The use of HMB to suppress proteolysis originates from the observations that leucine has protein-sparing characteristics (18; 24). The essential amino acid leucine can either be used for protein synthesis or transaminated to the α-ketoacid (α-ketoisocaproate, KIC) (24). In one pathway, HMB is formed in the liver via oxidation of the leucine transamination product α-ketoisocaproate. Approximately 5% of leucine oxidation proceeds via this pathway (28). HMB is superior to leucine in enhancing muscle mass and strength. The optimal effects of HMB can be achieved at 3.0 grams per day, or 0.038 g/kg of body weight per day, while those of leucine require over 30.0 grams per day (29).

Once produced or ingested, HMB appears to have two fates. The first fate is simple excretion in urine. After HMB is fed, urine concentrations increase, resulting in an approximate 20-50% loss of HMB to urine (26; 52). Another fate relates to the activation of HMB to HMB-CoA (4; 6; 16; 17; 20; 35; 36; 41; 43; 54). Once converted to HMB-CoA, further metabolism may occur, either dehydration of HMB-CoA to MC-CoA, or a direct conversion of HMB-CoA to HMG-CoA (42), which provides substrates for intracellular cholesterol synthesis. Several studies have shown that HMB is incorporated into the cholesterol synthetic pathway (2-4; 16) and could be a source of cholesterol for new cell membranes that are used for the regeneration of damaged cell membranes (29). Human studies have shown that muscle damage following intense exercise, measured by elevated plasma CPK (creatine phosphokinase), is reduced with HMB supplementation. The protective effect of HMB has been shown to manifest itself for at least three weeks with continued daily use (14; 22; 23).

In vitro studies in isolated rat muscle show that HMB is a potent inhibitor of muscle proteolysis (32) especially during periods of stress. These findings have been confirmed in humans; for example, HMB inhibits muscle proteolysis in subjects engaging in resistance training (26). The results have been duplicated in many studies (14; 22; 33; 46; 53) (9-11; 47; 48; 48) In C2C12 muscle cells, HMB attenuates experimentally-induced catabolism (e.g.

The molecular mechanisms by which HMB decreases protein breakdown and increases protein synthesis have been reported (10; 44). In mice bearing the MAC16 cachexia-inducing tumor, HMB attenuated protein degradation through the down-regulation of key activators of the ubiquitin-proteasome pathway (47). Furthermore, HMB attenuated proteolysis-inducing factor (PIF) activation and increased gene expression of the ubiquitin-proteasome pathway in murine myotubes, thereby reducing protein degradation (48). PIF inhibits protein synthesis in murine myotubes by 50% and HMB attenuates this depression in protein synthesis (9). Eley et al demonstrated that HMB increases protein synthesis by a number of mechanisms, including the down-regulation of eukaryotic initiation factor 2 (eIF2) phosphorylation through an effect on dsRNA-dependant protein kinase (PKR) and upregulation of the mammalian target of rapamycin/70-kDa ribosomal S6 kinase (mTOR/p70^{S6k}) pathway. The net result is increased phosphorylation of 4E-binding protein (4E-BP1) and an increase in the active eIF4G.eIF4E complex. Leucine shares many of these mechanisms with HMB, but HMB appears to be more potent in stimulating protein synthesis (9).

HMB can also increase protein synthesis by attenuating the common pathway that mediates the effects of other catabolic factors such as lipopolysaccharide (LPS), tumor necrosis factor-α/interferon-γ (TNF-α/IFN-γ), and angiotensin II (Ang II) (10; 11). HMB acts by attenuating the activation of caspases-3 and -8, and the subsequent attenuation of the activation of PKR and reactive oxygen species (ROS) formation via down-regulation of p38 mitogen activated protein kinase (p38MAPK). Increased ROS formation is known to induce protein degradation through the ubiquitin-proteasome pathway. HMB accomplishes this attenuation through the autophosphorylation PKR and the subsequent phosphorylation of eIF2a, and in part, through the activation of the mTOR pathway.

A recent report tested the hypothesis that HMB, like leucine, would stimulate mTORc1 independent of PI3K signaling in C2C12 myotubes (19). HMB stimulated the phosphorylation of AKTSer473 (+129%), S6K1Thr389 (+50%) and 4EBP1Thr65/70 (+51%). HMB stimulated anabolic signaling with greater potency than leucine, e.g. S6K1Thr389+50% vs. +17%; respectively. As expected, incubation of HMB with rapamycin (mTORc1 inhibitor) ablated increases in mTORc1 signaling, but not AKT phosphorylation (+188%). In contrast, incubation with LY290042 (PI3K inhibitor) abolished HMB-induced increases in both AKT and mTORc1 signaling, suggesting HMB signals to mTORc1 in a PI3K-dependent manner. These data suggest that HMB, despite being a leucine metabolite, signals to mTORc1 through mechanisms distinct from those of leucine.

Numerous studies have shown an effective dose of HMB to be 3.0 grams per day as CaHMB (˜38 mg/kg body weight-day⁻¹). As a dietary supplement, HMB has been used as the mono-hydrated calcium salt, whose empirical formula is Ca(HMB)₂—H₂0. This dosage increases muscle mass and strength gains associated with resistance training, while minimizing muscle damage associated with strenuous exercise (14; 26; 30; 33). HMB has been tested for safety, showing no side effects in healthy young or old adults (15; 25). HMB in combination with L-arginine and L-glutamine has also been shown to be safe when supplemented to AIDS and cancer patients (38).

Studies in humans have also shown that dietary supplementation with 3.0 grams of CaHMB per day plus amino acids attenuates the loss of muscle mass in various conditions such as cancer and AIDS (5; 12). A meta-analysis of supplements to increase lean mass and strength with weight training showed HMB to be one of only 2 dietary supplements that increase lean mass and strength with exercise (30). More recently it was shown that HMB and the amino acids arginine and lysine increased lean mass in a non-exercising, elderly population over a year-long study.

Leucine oxidation increases after exercise, and optimal levels of HMB during and just after exercise would be desired for optimal prevention of muscle damage and subsequent recovery. Further, the inflammatory process is stimulated during an injury, which if left unchecked is deleterious and delays healing. Chronic inflammation and pro-inflammatory cytokines have been shown to be a major underlying and causative factor in cardiovascular disease and type II diabetes, as well as in asthma, autoimmune diseases, inflammatory bowel disease, chronic obstructive pulmonary disease and rheumatoid arthritis.

Human studies have shown a positive effect of resistance exercise on muscle protein synthesis as early as 1-2 hours post exercise and lasting up to 48 hours (8; 34). Studies have also shown that the timing of nutrient availability to be critical for maximal post-exercise stimulation of protein synthesis as well as blunting of protein breakdown (40). The most optimal time for delivery of nutrient appears to be within 2 hours post-exercise. Dreyer at al. (7) demonstrated that the ingestion of a leucine-rich nutrient solution within 1 h of post-exercise recovery resulted in significant enhancement of the mTOR signaling pathway and muscle protein synthesis.

The dissociation curve of CaHMB is identical to that of calcium acetate (49) resulting in peak plasma HMB levels ranging from 60 to 120 minutes after ingestion depending upon the dosage given. Time to peak plasma levels after a typical 1 gram dosage was 2 hours (52), thus requiring CaHMB to be taken before exercise for maximal benefit.

Thus, the timing of HMB administration and the HMB level in the blood are important to the efficacy of HMB on muscle. The need exists for a faster and more efficient delivery system for HMB.

CaHMB is the mono-hydrated calcium salt combined with HMB to create a powder. HMB free acid (HMB-acid) is HMB in its free acid form. HMB-acid is more rapidly absorbed than CaHMB resulting in a higher peak and sustained concentration in blood, which maximizes the results on muscle obtained through the use of HMB. HMB-acid increases clearance compared to CaHMB, which results in greater retention and more HMB being available to muscle tissue. HMB-acid has improved efficacy over CaHMB for increasing strength gains.

It is desirable to increase the oral bioavailability of HMB to increase the extent of the effect on the user. It has surprisingly and unexpectedly been found that administration of HMB in the free acid form (HMB-acid) increases the oral bioavailability of HMB and thus improved efficacy following administration in an animal.

### SUMMARY OF THE INVENTION

One object of the present invention is to provide a composition and methods of using the composition for enhancing and/or increasing the bioavailability of HMB.

Another object of the present invention is provide a composition of HMB-acid and methods of using the composition to increase the area under the curve (AUC) of HMB as compared to administration of CaHMB.

A further object of the present invention is to provide a composition and methods of using the composition of HMB-acid to increase the peak plasma concentration of HMB as compared to CaHMB.

An additional object of the present invention is to provide a composition and methods of using the composition of HMB-acid to improve tissue utilization of HMB.

Another object of the present invention is to provide a composition and methods of using the composition of HMB-acid to increase the availability of HMB to tissues.

A further object of the present invention is to provide a composition and methods of using the composition of HMB-acid to minimize muscle tissue damage more effectively and to a greater extent than a similar dose of CaHMB.

An additional object of the present invention is to provide a composition and methods of using the composition of HMB-acid to improve muscle function, including muscle strength, when HMB-acid is used as compared to a similar dosage of CaHMB.

Another object of the present invention is to provide a composition and methods of using the composition of HMB-acid to improve muscle performance and/or increase muscle mass to a greater degree than when a similar dosage of CaHMB is used.

The invention is administration of HMB in a free acid form (“HMB-acid”). Administration using HMB free acid improves HMB availability to tissues and thus provides a more rapid and efficient method to get HMB to the tissues than administration of CaHMB. The oral intake or sublingual administration of a free acid HMB associated with a matrix results in direct and rapid absorption of HMB, offering an improved method of delivery resulting in an increased availability of HMB to the tissues.

Methods are disclosed for increasing the oral bioavailability of HMB comprising administering HMB in the free acid (HMB-acid) form instead of the calcium HMB (CaHMB) form.

A method for improving absorption and systemic utilization of HMB is described, comprising administering to an animal an effective amount of HMB-acid.

In one example, the HMB free acid is delivered directly by neutralizing HMB free acid in a soluble matrix such as a gel. The HMB free acid is administered orally or sublingually to a person in an effective amount. In another example, the HMB free acid is administered orally via a capsule or softgel. In an additional example, the HMB free acid is administered orally via a liquid.

### BRIEF DESCRIPTION OF THE FIGURES

FIG. 1 shows plasma levels of CPK and LDH after a strenuous exercise bout.

FIG. 2 shows muscle strength and subjective soreness after a strenuous bout of exercise.

FIG. 3 shows plasma HMB levels as found in Experimental Example 1.

FIG. 4 shows peak plasma HMB concentration and time to peak concentration.

FIG. 5 shows plasma HMB levels.

FIG. 6 shows peak plasma HMB concentration and time to peak concentration.

FIG. 7 shows percent of HMB dosage excreted in the urine.

FIG. 8 shows a treatment regime of the present invention.

FIG. 9 shows changes in CPK after an acute bout of eccentric exercise.

FIG. 10 shows peak plasma HMB concentration and time to peak concentration with administration of CaHMB.

FIG. 11 shows peak plasma HMB concentration and time to peak concentration with administration of HMB-acid.

FIG. 12 shows the net percent increase in muscle function and performance measurements with HMB-acid compared to CaHMB administration.

### DETAILED DESCRIPTION OF PREFERRED EMBODIMENTS

The present invention represents a surprising finding that a dosage of HMB free acid exhibits an improved bioavailability over its calcium salt counterpart. The present invention includes the unexpected and surprising finding that HMB-acid has an improved pharmokinetic profile over CaHMB. The improved bioavailability and/or pharmokinetic profile of HMB-acid results in improvements in HMB availability and efficacy to tissues, resulting in greater improvements in muscle function, including muscle strength, greater gains in muscle mass, greater minimization of muscle damage, and/or greater utilization of HMB by skeletal muscle as compared to a similar dose of CaHMB.

Administering HMB in the free acid form is superior to the existing available form of CaHMB because HMB-acid has improved bioavailability over CaHMB. The improved bioavailability of HMB-acid results in better utilization of HMB by muscle tissues. Enhanced utilization of HMB by muscle tissues results in greater gains and improvements in muscle function, muscle strength, muscle performance and/or muscle mass. A person seeking to improve and/or increase muscle function, muscle strength, muscle performance and/or muscle mass would select the form that results in greater improvements in these physiological effects, especially when the doses uses remain substantially similar between the two forms. In other words, a person looking to improve and/or increase muscle function, muscle strength, muscle performance and/or muscle mass would prefer to use the intervention, nutraceutical or supplement that has better results at similar doses.

The invention is a method of delivery of HMB to a person, and specifically a method of administering HMB-acid to a person such that the administration of free acid HMB results in an increase in effectiveness of HMB over administration of other forms of HMB, including CaHMB. Use of HMB-acid results in the improvement of HMB availability to a person's tissues. The administration of HMB-acid increases tissue utilization of HMB, resulting in increased effectiveness for protecting against muscle damage and accompanying inflammatory response over administration of HMB in its other forms. Further, administration of free acid HMB may also preserve muscle in cachectic and wasting conditions and act to blunt inflammation, including chronic inflammation that may cause a number of diseases, such as cardiovascular disease as compared to administration of CaHMB. The unexpected and surprising discovery that free acid HMB decreases muscle damage better than CaHMB indicates that it may also decrease inflammatory response resulting from the damage. The administration of HMB free acid has widespread applications as a nutritional or medical supplement and may affect a large portion of the population.

Ingestion of HMB promotes muscle function, including improvements in muscle strength, endurance, recovery and increases in lean tissue. The present invention is directed at use of HMB-acid to enhance these and other benefits of ingestion of HMB as compared to use of CaHMB.

The present invention includes improving and/or enhancing the bioavailability of HMB. Bioavailability is the availability of the active ingredient to tissues, or the rate and extent to which the active ingredient is absorbed and becomes available at the site of action. As will be understood by one skilled in the art, for any and all purposes, particularly in terms of providing a written description, all ranges disclosed herein, and in particular bioavailability ranges described herein also encompass any and all possible subranges and combinations of subranges thereof. Bioavailability may be achieved by at least about 10%, at least about 20%, at least about 30%, at least about 50%, up to 100% or even 200%. Pharmokinetic measures of bioavailability include the area under the curve (AUC), peak concentration, time to peak concentration, and clearance rate. Typically bioavailability is asses by measuring the concentration of HMB at various points of time after administration of HMB. AUC is a direct measurement of the bioavailability of HMB.

The improved bioavailability of HMB to tissues through the use of HMB-acid results in increased HMB availability and efficacy to tissues and/or utilization by tissues, including muscle tissue, which results in improvements in muscle function, including muscle strength and increases in muscle mass, as compared to a similar dose of CaHMB. Improvements in muscle function, including strength, and/or muscle mass include achievement of increases in function, strength, and/or mass by at least about 10%, at least about 20%, at least about 30%, at least about 50%, at least about 60%, at least about 70%, at least about 70%, at least about 80%, at least about 80%, up to 100% or even 200%. Measures of improved efficacy and/or utilization include improved and/or increased muscle function, muscle strength, and/or muscle mass.

HMB-acid produces significantly higher plasma levels of HMB indicating improved absorption of the HMB-acid form over the calcium salt (CaHMB) form.

In accordance with the present invention, HMB is administered to humans in its free acid form. The free acid HMB may be associated with a carrier, such as a matrix or gel. In the preferred embodiment, the free acid HMB is administered orally or sublingually, although any means of administering HMB is appropriate.

As used herein, an “effective amount” of compositions of the present invention is defined as an amount effective, at dosages and for periods of time necessary, to achieve the desired result. The effective amount of compositions of the invention may vary according to factors such as age, sex, and weight of the individual. Dosage regime may be adjusted to provide the optimum response. Several divided doses may be administered daily, or the dose may be proportionally reduced as indicated by the exigencies of an individual's situation. As will be readily appreciated, a composition in accordance with the present invention may be administered in a single serving or in multiple servings spaced throughout the day. As will be understood by those skilled in the art, servings need not be limited to daily administration, and may be on an every second or third day or other convenient effective basis. The administration on a given day may be in a single serving or in multiple servings spaced throughout the day depending on the exigencies of the situation.

The term administering or administration includes providing a composition to a mammal, consuming the composition and combinations thereof.

The compositions according to the present invention may be employed in methods for supplementing the diet of an individual, e.g., an athlete, an elderly person, etc. and/or for enhancing an individual's muscle mass and/or muscle size and/or strength, and/or endurance. Accordingly, the present invention provides methods of supplementing the dietary intake of an individual comprising administering to the individual an effective amount of a composition (e.g., HMB-acid or a nutritional supplement comprising HMB-acid) according to the present invention to increase muscle performance or muscle function is said individual. The invention also relates to methods of improving muscle performance and/or muscle function, including muscle strength in an individual comprising administering an effective amount of HMB-acid (alone or in combination with other agents, e.g., in a nutritional supplement) to the individual.

While it is expected that the compositions and methods of the present invention will be of particular importance to bodybuilders and other athletes, the usefulness of compositions and methods of the invention is not limited to those groups. Rather, any individual may beneficially use the compositions and methods of the invention. Indeed, the disclosed compositions and methods have application to all animals, including mammals, birds and reptiles. As used herein, the term “animal” includes all members of the animal kingdom, preferably mammals (e.g., dogs, horses, cows, mules), more preferably humans. For example, the nutritional supplements of the invention may have beneficial effect for competitive animals (e.g., racehorses, show horses, racing dogs (e.g., greyhounds), bird dogs, show dogs) and work animals (e.g., horses, mules and the like) in whom an increase in muscle function is desirable.

The nutritional supplement compositions according to the present invention can further comprise one or more acceptable carriers. A wide number of acceptable carriers are known in the nutritional supplement arts, and the carrier can be any suitable carrier. The carrier need only be suitable for administration to animals, including humans, and be able to act as a carrier without substantially affecting the desired activity of the composition. Also, the carrier(s) may be selected based upon the desired administration route and dosage form of the composition. For example, the nutritional supplement compositions according to the present invention are suitable for use in a variety of dosage forms, such as liquid form and solid form (e.g., a chewable bar or wafer). In desirable embodiments, as discussed below, the nutritional supplement compositions comprise a solid dosage form, such as a capsule or softgel. Examples of suitable carriers for use in tablet and capsule compositions include, but are not limited to, organic and inorganic inert carrier materials such as gelatin, starch, magnesium stearate, talc, gums, silicon dioxide, stearic acid, cellulose, and the like.

The compositions may contain pharmaceutically, e.g., nutraceutically, acceptable excipients, according to methods and procedures well known in the art. As used herein, “excipient” refers to substances that are typically of little or no therapeutic value, but are useful in the manufacture and compounding of various pharmaceutical preparations and which generally form the medium of the composition. These substances include, but are not limited to, coloring, flavoring, and diluting agents; emulsifying, dispersing and suspending agents, ointments, bases, pharmaceutical solvents; antioxidants and preservatives; and miscellaneous agents.

As used herein, the terms “nutraceutical” and nutraceutically acceptable” are used herein to refer to any substance that is a food or part of a food and provides medical or health benefits, including the prevention and treatment of disease. Hence, compositions falling under the label “nutraceutical” or “nutraceutically acceptable” may range from isolated nutrients, nutritional or dietary supplements, and specific diets to genetically engineered designer foods, herbal products, and processed foods such as cereals, soups, and beverages. In a more technical sense, the term has been used to refer to a product isolated or purified from foods, and generally sold in medicinal forms not usually associated with foods and demonstrated to have a physiological benefit or provide protection against chronic disease.

HMB in its acid form is called 3-hydroxy-3-methylbutyric acid, β-hydroxy-β-methylbutyric acid, or β-hydroxy-isovalaryic acid and can be designated “HMB-acid.” The structural formula is (CH₃)₂C(OH)CH₂COOH and the molecule is:

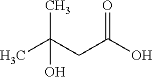

In the present invention, HMB-acid is administered to a human in an effective amount. An effective amount includes a range from about 0.01 grams to about 0.2 grams of HMB-acid per kilogram body weight in twenty-four (24) hours. HMB-acid may also be administered to a human in an effective amount from about 0.5 grams to about 30 grams of HMB-acid per day. An effective amount of HMB-acid will result in a greater increase in plasma levels of HMB and/or will result in a faster time to reach peak plasma levels of HMB relative to administration of a similar dosage of CaHMB The increase in the effectiveness with administration of HMB-acid may be 10%, 20%, 30%, 50%, 75%, 100%, 200%, 400%, 500% or greater than administration of a similar dose of CaHMB. Comparison of HMB-acid with other forms of HMB may be based on effectiveness or efficiency of HMB using standard indices known to those of skill in the art.

In the some of the Examples, the HMB-acid is administered as a soluble gel, although the invention is not limited to use of a soluble gel or matrix with HMB-acid. HMB-acid in any pharmaceutically acceptable form, including but not limited to solids, liquids, tablets, capsules (including softgel capsules and/or hard shell capsules), and liquids such as oral intravenous solutions, is within the scope of this invention. The HMB-acid can be administered utilizing any pharmaceutically acceptable carrier, including but not limited to various starches and saline solutions. In the preferred embodiment, an effective amount of HMB-acid is administered as two or three daily doses, although a single dose of an effective amount of HMB-acid per day will be understood to be within the scope of the invention, as would be any other number of doses of HMB during the day.

The delivery of HMB-acid, most typically as a HMB-acid soluble matrix such as a gel, results in a significant improvement in HMB's anabolic effect with marked reductions in CPK over administration of HMB as a salt, including CaHMB or administration of HMB in other forms such as an ester or lactone. In one embodiment, the administration of HMB-acid gel results in a doubling of the plasma peak of HMB in about ¼ of the time as administration of a similar dosage of CaHMB, and has a 25% improved efficiency of delivery as measured by plasma clearance over a similar dosage of CaHMB.

This method of delivery has widespread applications. HMB is used as a medical, pharmaceutical, nutraceutical or dietary supplement in many different populations, including athletes, bodybuilders, and those in need of improving muscle mass, muscle function and/or muscle strength, including the elderly. Known uses or benefits of HMB include, but are not limited to, improved nitrogen retention and protein sparing, improving lean body mass, improving muscle function and/or muscle performance (including improving muscle strength), decreasing muscle damage in muscle subjected to stress or damage, decreasing inflammatory response after muscle is subjected to stress or damage, improving the body's immune response after stress or damage, treating disease associated wasting (such as wasting associated with cancer, chronic pulmonary disease, age, chronic kidney disease, long-term hospitalization or AIDS), improving a lipid profile such a low density lipoprotein (LDL) to high density lipoprotein (HDL), and improving a person's emotional state.

A more effective and more efficient way to administer HMB has widespread applications in all of these known uses of HMB. Increased availability of HMB to tissues with administration of HMB-acid results in improved efficacy and/or efficiency of HMB; the increased utilization of HMB by tissues results in improved effects of administering HMB such as improved nitrogen retention and protein sparing, improving lean body mass, improving muscle function and/or muscle performance (including improving muscle strength), decreasing muscle damage in muscle subjected to stress or damage, decreasing inflammatory response after muscle is subjected to stress or damage, improving the body's immune response after stress or damage, treating disease associated wasting (such as wasting associated with cancer, chronic pulmonary disease, age, chronic kidney disease, long-term hospitalization or AIDS), improving a lipid profile such a low density lipoprotein (LDL) to high density lipoprotein (HDL), and improving a person's emotional state as compared to administration of CaHMB. One of skill in the art understands that use of HMB-acid and the resultant increase in HMB availability to tissue will improve any known effects of administering HMB as compared to administration of CaHMB.

While use of HMB-acid has previously been stated, HMB in free acid form was thought to be equivalent to HMB in the calcium salt and other salts as proposed administration forms in the prior art. Differences in the effectiveness of HMB-acid and HMB salts were not previously tested. It was not known or suggested by the literature that the HMB-acid form of HMB would exhibit improved effects, including improved effects on muscle function, muscle strength, and/or muscle mass over using a similar dose of CaHMB. People who use pharmaceutical, nutraceutical and/or dietary compositions to improve their health prefer an optimized form, wherein the optimal form confers greater physiological benefits (such as greater improvements in muscle function, muscle strength, and/or muscle mass) when taken in similar amounts to a less optimal form. In this specific instance, consumers of HMB would prefer to use the form with improved bioavailability to result in greater strength gains, greater improvements in muscle function and/or greater increases in muscle mass. A method of optimizing the benefits conferred by using HMB had not been measured or determined until the present inventors discovered the significant differences in the bioavailability and thus the efficacy on muscle between CaHMB and HMB-acid. When choosing between forms of HMB to use, a person will choose the form that confers greater physiological benefits at similar doses to the alternative.

Even though it was thought that HMB-acid and CaHMB would confer similar benefits to a person consuming them, and the literature even suggests that CaHMB would be absorbed better than HMB-acid, the present inventors have made the surprising and unexpected discovery that HMB-acid exhibits significantly increased bioavailability to muscle tissues, resulting in longer periods of time that muscle tissue is exposed to HMB. The longer exposure times to HMB result in improved physiological results, including increased gains in strength and mass, improvements in muscle function, and improvements in muscle performance as compared to the shorter exposure times that result from consuming a similar dose of CaHMB. The HMB-acid form of HMB exhibits enhanced bioavailability. Exposing tissues to higher levels of HMB is highly desirable. The methods of improving the efficacy of HMB by delivering HMB to muscle by using HMB-acid is far superior to prior known methods, including using CaHMB, and this result was not predictable.

Administering HMB-acid to a person confers one or more of the following physiological effects: increased skeletal muscle levels of HMB, increased muscle performance, increased muscle strength, improved muscle function, and/or increased muscle mass as compared to use of a similar dose of CaHMB.

In all instances known to the inventors, HMB utilized in clinical studies and marketed as an ergogenic acid has been in the calcium salt form due to significant issues with developing HMB-acid for animal consumption. Previously, numerous obstacles existed to both extensive testing and commercial utilization of the free acid form of HMB, and since it was thought there was no difference, the calcium salt was adopted as a commercial source of HMB. Until recently packaging and, in particular, distribution of dietary supplements has been better suited to handle nutrients in a powdered form and therefore the calcium salt of HMB was widely accepted. HMB-acid is a liquid and much more difficult to deliver or incorporate into products. Further, while HMB-acid has been previously described, HMB-acid was only available as a raw ingredient and had not been prepared as a pharmaceutical formulation or as a food grade ingredient.

Unlike other calcium salts, it had been shown that the calcium and HMB components of the molecule dissociate very easily, therefore, adding to the assumption that there would be no physiological difference between HMB free acid and the calcium salt of HMB (19) . . . (49).

Additionally, formation and crystallization of the calcium salt of HMB had been utilized as a final purification stage in the manufacturing process. One compound in particular that the crystallization served to limit was 3,3-Dimethyl acrylic acid. This compound adds a very off flavor which is hard to mask. Currently, the manufacturing process for HMB has allowed for HMB free acid to be produced in a purity that allows for oral ingestion of the HMB free acid; the present invention utilized this newly developed, purified food-grade and pharmaceutical formulation of HMB-acid. Food grade, dietary supplement, nutraceutical or pharmaceutical formulations of HMB-acid have not been described prior to the present invention.

HMB-acid can be buffered using physical or chemical buffers. Examples of physical buffers include but are not limited to encapsulating the HMB-acid, including use of capsule such as hard-shell or softgel capsules, preparing the HMB-acid with a gel matrix delivery form, and/or incorporating the HMB-acid into a liquid, including nutritional liquids or beverages.

Preferred methods of administration included oral routes. Additional ingredients, including but not limited to protein, amino acids, vitamins, minerals, carbohydrates, starches, thickening agents, emulsifying agents, sweeteners and flavorings may be included with HMB-acid. Typically HMB-acid is provided as a nutritional and/or dietary supplement that is orally ingested, but it may also be administered via other routes.

Because the calcium and HMB in the calcium salt was loosely associated (49), it was previously thought that there would be no difference in oral administration of HMB either as a free acid or as a calcium salt (19). As shown in Example 1, not only is there a surprising difference in plasma levels attained with oral administration of HMB-acid, but there is a 25% increase in plasma clearance which indicates increased utilization of HMB by tissues with resultant improved effects on muscle mass and function, including strength. When given in molar equivalents, HMB in free acid form results in double the plasma level of HMB in about one fourth (¼) the time when compared to the calcium salt of HMB. The improved effect on muscle is clearly shown in Example 2 in that HMB administered in free acid form is more protective than CaHMB when muscle is subjected to acute exercise.

While it is known that CaHMB is soluble in and easily dissociates in aqueous solutions of neutral to acidic pH, one of skill in the art would predict a short lag time in appearance due to this dissociation step. CaHMB administered in a gelatin capsule has a dissolution time of between 10 and 15 minutes in the gut. Therefore, one skilled in the art would expect similar absorption and thus peak plasma levels of HMB whether the HMB was administered as CaHMB or as HMB-acid, however, slightly delayed for the differences described. It has even been suggested that the salt or chelate forms of HMB, including CaHMB are preferable forms because these forms aid absorption. See Sparkman (US Patent Application Publication No. 2008/0317886. Contrary to these expectations (and the opposite of the theory described in Sparkman), however, it was found that there was a significant difference in plasma peak between the two forms. In some instances, the difference in peak plasma times was ninety (90) minutes, which is several fold longer than should be accounted for by capsule dissolution and disassociation of the CaHMB.

One skilled in the art would also predict similar plasma peaks and areas under the curve since a similar amount of the nutrient HMB was being released into the gut with the CaHMB curve shifted further out in time. An unexpected result of administering HMB-acid is the doubling in peak plasma HMB levels.

An additional unexpected result is the greater clearance (utilization) of HMB once in the plasma. Many nutrients have similar plasma and urinary profiles unless there is a conservation mechanism in the kidney for that nutrient. None is presently known for HMB and thus one skilled in the art might presume a much higher percentage of the dosage would be excreted in the urine with the doubling of the plasma peak in HMB concentrations. Again, this was not observed and is an unexpected finding. This coupled with the higher clearance rate show improved utilization of HMB by tissues which again was a surprising finding.

As shown in the Experimental Examples, and specifically in Example 2, administering HMB-acid is more quickly effective in minimizing muscle damage after exercise than CaHMB. This second example shows a benefit that could not have been predicted directly from the findings of Example 1.

The method of this invention is further illustrated by the following experimental examples.

### EXPERIMENTAL

### Example 1: Absorption of HMB-Acid Gel Compared with CaHMB in Capsule Form

### Materials and Methods

Human subjects. In study 1, four male and four female college-aged subjects were studied. In study 2, an additional four male and four female college-aged subjects were studied. The protocols for both studies were approved by the Iowa State University IRB and each subject gave informed consent to participate in the study. Due to the nature of the treatments, neither the subjects nor the researchers could be blinded.

Treatments. The same treatments were given to the subjects in both study groups. The three treatments were given in random order to each subject with at least a one week washout period between treatments. The treatments were supplied by Metabolic Technologies, Inc. (MTI, Ames, Iowa) and were prepared with food grade ingredients. One gram of CaHMB or the equivalent HMB in free acid in a gel form was administered to the subjects. The CaHMB capsules were obtained from a commercial supplement manufacturer (Optimum Nutrition, Aurora, Ill.) while the HMB-acid gel was prepared at MTI laboratories. Briefly, the HMB-acid was adjusted to a pH 4.5 with potassium carbonate (K₂CO₃) and flavors and sweeteners were then added. The 1.0 g CaHMB capsule was taken with 355 mL of water (approx. 12 oz.). The free acid gel dosage was 0.80 g and was equivalent to the free acid contained in the CaHMB in the capsule. The free acid gel treatments were either swallowed (FASW) or held sublingual for 15 seconds and then swallowed (FASL). FASW consisted of expelling the entire dose into the mouth in a 3 ml syringe, swallowing, and then following this with 355 mL of water. FASL subjects were instructed to place the entire dosage under the tongue and hold the dosage for 15 sec before swallowing. They then rinsed and followed the dose with 355 mL of water.

Study 1 design. For study 1 the subjects reported to the laboratory in the morning following an overnight fast. Before ingestion of one of the supplemental treatments, a flexible sterile polyethylene catheter was inserted into a forearm vein using sterile procedures and a pre-ingestion blood sample was drawn. Subsequent blood samples were taken at 0, 2, 5, 10, 15, 25, 35, 45, 60, 90, 120 and 180 min after ingestion of the treatment. Plasma was separated and samples were stored frozen at −70° C. for analysis of HMB concentration. In addition, a portion of the pre-ingestion and 180 minute blood samples were used for measurements (LabCorp, Kansas City, Mo.) of glucose, uric acid, blood urea nitrogen (BUN), creatinine, sodium, potassium, chloride, carbon dioxide, phosphorous, protein, albumin, globulin, albumin:globulin ratio, total bilirubin, direct bilirubin, alkaline phosphatase, lactate dehydrogenase, aspartate aminotransferase (AST), alanine aminotransferase (ALT), gamma-glutamyl transpeptidase (GGT), iron binding capacity (TIBC), unsaturated iron binding capacity (UIBC), iron, iron saturation, total cholesterol, triglycerides, high density lipoprotein (HDL), low density lipoprotein (LDL), and cholesterol ratio. A complete blood count (CBC) was also performed before and after the 180 min treatment period. Subjects also completed a brief questionnaire to report any physical symptoms (such as nausea, headache, etc) they may have experienced during the experiment.

Study 2 design. Study 2 was conducted similar to study 1 with the following modifications. In study 2, plasma levels of HMB were measured for 1440 min (24 h) and total urine collection was also performed for measurement of urinary HMB excretion during this period. After the 180 min blood sample, the subjects were allowed to leave the laboratory and were instructed to return to the laboratory for additional blood samples at 360, 720, and 1440 min after the ingestion of the supplement. As in study 1, each subject took each of the treatments with at least a one week washout period between treatments. Samples were again stored frozen at −70° C. for analysis of plasma and urinary HMB concentrations. Pre-ingestion, 180 min and 1440 min blood samples were again assayed for the same measurements already for study 1. Subjects were provided with a standardized lunch after the 180 min blood sampling and instructed to eat this at approximately 240 min post ingestion. Following the 720 min blood sample, subjects were instructed to eat a normal evening meal before 10 pm. Subjects reported back to the laboratory the following morning for the fasted 1440 min blood sample. A urine collection container was provided and subjects collected all urine produced during the experimental 24 h experimental period. The urine was stored refrigerated when not being collected. Urine volumes were measured and samples of the total urine collection were taken and stored frozen at −70° C. until analyzed for HMB.

HMB analysis. Plasma and urine HMB were analyzed by gas chromatography/mass spectrometry (GC/MS) as previously described.(27)

Calculations and statistics. Areas under the curves were calculated for each subject using the trapezoidal method that sums the area above baseline.(51) Half-life of plasma HMB was calculated for study 2. The following equations were used:

k=(ln(Cₚₑₐₖ)−ln(C_{trough}))/Tᵢₙₜₑᵣᵥₐₗ

t_{1/2}=0.693/k

Peak plasma concentrations for each subject were used for Cₚₑₐₖ. Trough concentrations, C_{trough} were the concentrations measured at 720 min, because the 720 min plasma concentrations were not significantly different from baseline. Tₚₑₐₖ was the time at which Cₚₑₐₖ was measured and Tᵢₙₜₑᵣᵥₐₗ is the time from Tₚₑₐₖ until the time at C_{trough} (720 min). The extracellular fluid compartment was assumed to be 20% of body weight and calculated using equation 3 below(1). The plasma clearance of HMB was then calculated by multiplying the extracellular fluid compartment, V_{d}, by the elimination constant, Kₑₗ, as shown in equation 4 {Thalhammer, 1998 9588/id}.

V_{d}=Body wt(0.20)

Clearance=V_{d}(Kₑₗ)(50).

The data were analyzed using a crossover design with Proc GLM in SAS. (45) For the timed sampling of plasma HMB a repeated measures polynomial model was used. The model included subject, order, and treatment main effects and time by treatment interaction where appropriate. For other parameters Proc GLM was also used with subject, order, and treatment main effects and the p values are reported for the treatment main effect. Statistical significance was determined for p<0.05 and a trend was determined for 0.05<p<0.10.

Results

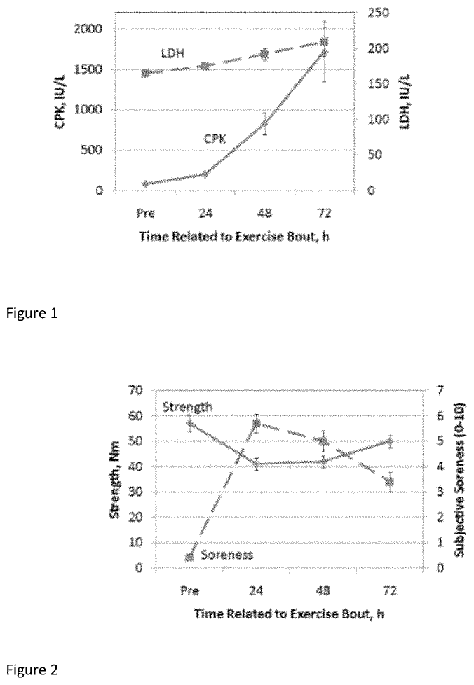

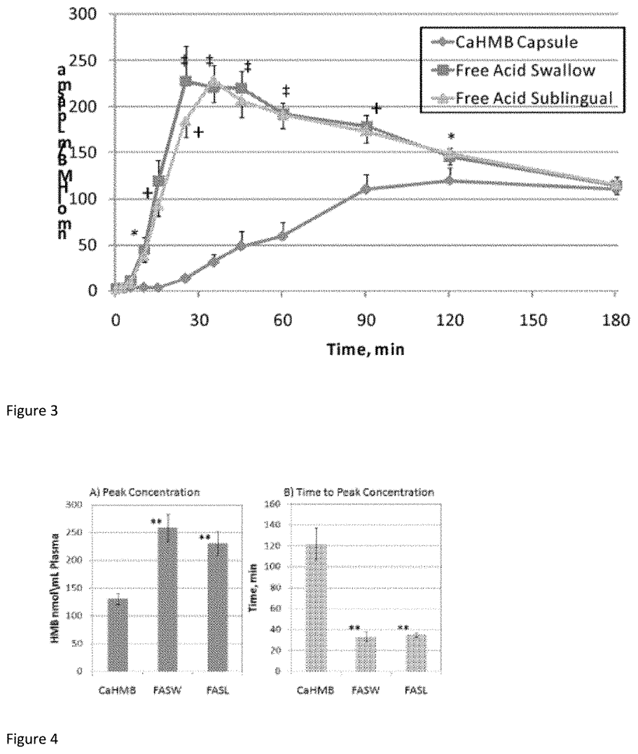

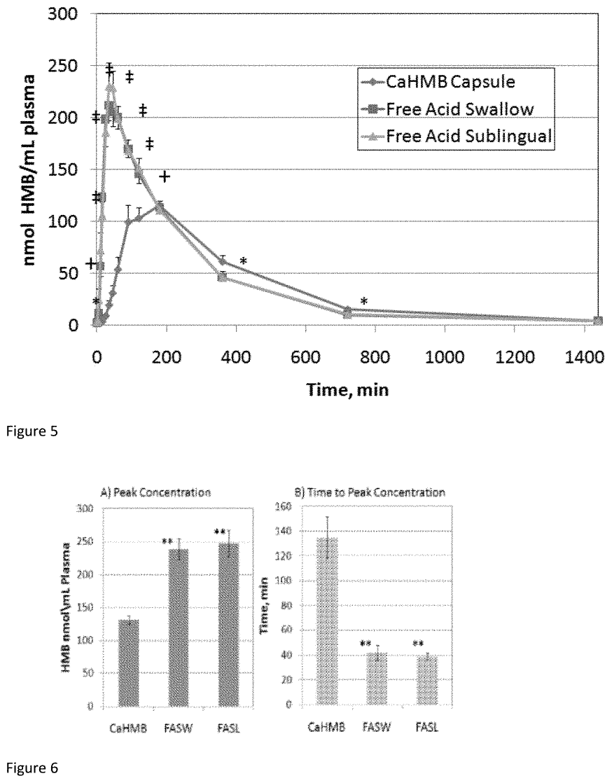

The results of the studies are shown in FIGS. 1-7. FIG. 1 shows plasma levels of CPK and LDH after a strenuous exercise bout. FIG. 2 shows muscle strength and subjective soreness after a strenuous bout of exercise. FIG. 3 shows plasma HMB levels in study 1. Values are means+/−SEM, n=4 males and 4 females. *=p, 0.05, +=p, 0.001, +p, 0.0001 for free acid treatments versus CaHMB capsules. FIG. 4 shows Study 1 peak plasma HMB concentration and time to peak concentration after a single dose of either CaHMB, HMB-acid gel swallowed (FASW, or HMB-acid gel held sublingual and then swallowed (FASL). **=p, 0.0002. FIG. 5 shows plasma HMB levels in study 2. Values are means+/−SEM, n=4 males and 4 females. *=p, 0.05, +=p, 0.01, ++p, 0.0001. FIG. 6 shows Study 2 peak plasma HMB concentration and time to peak concentration after a single dose of either CaHMB, HMB free acid gel swallowed (FASW, or HMB free acid gel held sublingual then swallowed (FASL). **=p<0.0002 for concentration and p<0.0001 for time. FIG. 7 shows percent of the HMB dosage excreted in the urine over 24 h post ingestion and relative percentage of the dose retained. *=p<0.002.

Subject characteristics. Table 1 shows the subject characteristics for studies 1 and 2. Each study was balanced for gender and each treatment group maintained steady weights throughout the 3 testing periods. In each study all subjects completed all 3 testing protocols. No adverse treatment effects, such as nausea after taking the treatments, were reported in either study.

Study 1 results (Tables 2-4). Table 2 illustrates the pharmokinetic parameters of peak plasma concentrations (Cₚₑₐₖ) and time to reach peak plasma concentrations (tₚₑₐₖ). CaHMB taken by capsule resulted in a peak plasma HMB level of 131±10 nmol/mL, whereas HMB taken either by free acid gel FASW or FASL delivery resulted in significantly greater (259±24 and 231±21 nmol/mL, respectively, p<0.0001) and earlier (33.1±4.6 and 36.3±1.3 min, respectively) peaks in plasma HMB levels compared with CaHMB by capsule at 121.9±15.6 h (p<0.0001). At 180 min, plasma HMB for all delivery methods was still elevated above baseline, and there were no treatment differences among the three groups. Consequently, the pharmokinetic parameter of areas under the curve (AUC) for plasma HMB levels following the 3 treatments are also shown in Table 2. HMB administered in free acid gel form resulted in 97% and 91% greater areas under the curve (AUC) for FASW and FASL, respectively (p<0.0001). There were no differences between HMB-acid gel delivered by FASW or by FASL.

Table 3 shows blood chemistries for study 1. There were no significant main effect treatment differences for any of the measured time points or differences. Table 4 shows blood hematology measured in study 1. The FASW group had significantly greater decrease in absolute lymphocyte numbers over the measurement period (p<0.04) due primarily to the fact that the FASW group tended to have higher lymphocyte numbers at the start of the study (p<0.09). There were no significant differences in lymphocyte numbers at the end of the study and all the means were within normal limits for lymphocyte number.

Study 2 results (Tables 2, 5-7). Study 2 was conducted to look at plasma HMB for a 24 hour period as well as to measure urinary losses during this same time period. Similar to study 1, a rapid increase in and significantly greater plasma HMB with HMB given in free acid gel form for both FASW and FASL than with CaHMB in capsule form was seen. At 180 min all treatments resulted in similar plasma HMB levels (approximately 110 nmol/mL). CaHMB by capsule did maintain a slightly higher plasma HMB level at 360 and 720 min (p<0.05).

Table 5 lists peak plasma HMB concentrations (Cₚₑₐₖ), time to peak plasma HMB concentrations (tₚₑₐₖ), and plasma HMB half-life. Plasma HMB level for CaHMB by capsule peaked at 131.2±6.0 nmol/mL at 135.0±17.0 min, while FASW and FASL resulted in greater HMB plasma peaks (p<0.0003) in shorter time (p<0.0001), 238.6±16.0 nmol/mL at 41.9±5.8 min and 247.6±19.8 nmol/mL at 38.8±2.6 min for FASW and FASL, respectively.

Plasma half-life as shown in Table 5 for CaHMB by capsule was 3.17±0.22 h. Half-lives for HMB FASW and HMB FASL were 2.50±0.13 and 2.51±0.14 h, respectively (p<0.004). Area under the curve (AUC) and urinary HMB measured in study 2 are also shown in Table 5. Over the 24 hours period AUC for HMB administered as the free acid gel was significantly greater by 15.4 and 14.3% for FASW and FASL, respectively, than for CaHMB administered in capsules (p<0.001). Despite the significant increase in peak plasma HMB and AUC, urinary HMB losses were not significantly greater for the free acid gel treatments; urinary HMB losses were 14.7±2.0, 17.8±2.9, and 17.2±2.5% of the initial dosage lost for CaHMB, FASW and FASL, respectively. There was an approximately 25% increase in HMB clearance with the free acid gel form compared with the CaHMB form (P<0.003).

Blood chemistries measured during study 2 are shown in Table 6 and blood hematology measured in study 2 is shown in Table 7. Statistical analysis of the change in parameters over the 24 h measurement period showed no significant statistical changes (p<0.05) for any of the chemistry or hematology values measured. There was a strong trend for a difference with chloride (p<0.06). FASL showed a greater increase over the period compared with FASW; however neither FASW nor FASL was significantly different from CaHMB. A trend was also seen with sodium where FASW showed a larger increase over the measurement period than FASW or CaHMB (p<0.07). Means for chloride and sodium over the study period were well within normal values.

### DISCUSSION

It is clear from the present studies that oral or sublingual administration of HMB in free acid gel form resulted in more rapid and sustained increases in plasma HMB than when compared HMB administered as the calcium salt (CaHMB) in a hard gelatin capsule. Free acid gel administration of HMB resulted in significant increases (average +14.8%) in the AUC for plasma HMB without any major changes in plasma HMB half-lives or urinary losses. Combined this resulted in significantly increased clearance of HMB and utilization by tissues that was 25% greater than that of the CaHMB form. The data show the improved efficiency of this form of delivery of HMB.

The findings from this study related to plasma kinetics of CaHMB delivery agree with those previously reported by Vukovich et al (52) There is congruence of data related to peak plasma levels, time to peak and plasma half lives, occurred despite significant differences in mode of delivery of CaHMB (four 250 mg capsules by Vukovich et al vs. 1 gram capsule in the present studies). Interestingly, the delivery of HMB as free acid in a gel form resulted in faster peak levels (nearly 90 minutes earlier with the use of the free acid form) but similar half lives, despite plasma levels reaching almost 2-fold those achieved with oral administration of CaHMB.

Several studies have supported the use of HMB as a nutritional supplement during exercise. HMB has been shown to decrease muscle protein and membrane breakdown (22; 23; 26) and to enhance protein synthesis. (10) It would therefore be advantageous to have high levels of plasma HMB during the exercise period, and to have HMB retention to be as good as or even better than those previously reported (26; 29; 52). In this regard, oral administration of CaHMB would need to be administered at least 2 hours before any serious stressful bout of exercise, whereas HMB free acid gel may be administered before the exercise bout and have an almost immediate effect.

Delivery of the free acid form of HMB was also associated with significantly higher retention of HMB. Administration of the free acid gel form resulted in a significant increase in AUC while not significantly increasing urinary excretion which would indicate more HMB retention and utilization by the tissues compared with the CaHMB form. The estimated amount of HMB retained was 25% greater with the HMB-acid gel compared with the CaHMB form based upon plasma clearance. Previous studies by Vukovich et al showed that the oral delivery of 3 grams of CaHMB resulted in plasma peak levels that were 3 times higher than those achieved with a 1 gram dose (52). Nissen et al demonstrated a dose-dependent response to oral administration of CaHMB given twice daily at either 1.5 or 3 grams per day. They demonstrated the optimal dose of CaHMB to be 3.0 grams per day resulting in decreased release of creatine kinase (CK), an indicator of muscle damage, and 3-methylhistine, an indicator of protein breakdown (26). Similar findings were reported by Gallagher who also showed that daily administration of 6 grams per day would be more beneficial than 3 grams per day (14). Taken together, the above studies indicate a benefit to having more HMB available to the muscles during exercise. Thus, the HMB-acid form of HMB allows for optimization of the delivery timing of the HMB dose to achieve maximum compliance and efficacy during exercise.

Based upon HMB's ability to increase lean mass with exercise, delivery of maximal HMB to tissues during and immediately post-exercise results in further increases in protein synthesis.

HMB delivery by free acid gel results in a faster and greater peak in HMB blood levels as well as equally sustained levels when compared with CaHMB administered in a capsule. This form of delivery is equally safe as those currently and previously (15; 25) found with oral administration of CaHMB.

TABLE 1
Subject Descriptors.

| | Treatment | | |
|---|---|---|---|
| | | HMB-Acid | HMB-Acid |
| | CaHMB | Gel | Gel |
| | Capsule | Swallow | Sublingual |
| | Study 1 | | |
| Gender, male/female | 4/4 | 4/4 | 4/4 |
| Age, y | 23.8 ± 1.3 | 23.8 ± 1.3 | 23.8 ± 1.3 |
| Body Weight, kg | | | |
| All | 68.5 ± 3.9 | 68.5 ± 3.8 | 68.6 ± 4.0 |
| Females | 63.2 ± 4.5 | 63.5 ± 4.5 | 63.5 ± 4.9 |
| Males | 73.7 ± 5.6 | 73.5 ± 5.6 | 73.8 ± 5.9 |
| | Study 2 | | |
| Gender, male/female | 4/4 | 4/4 | 4/4 |
| Age, y | 22.4 ± 1.0 | 22.4 ± 1.0 | 22.4 ± 1.0 |
| Body Weight, kg | | | |
| All | 72.0 ± 4.2 | 72.4 ± 4.1 | 72.3 ± 4.1 |
| Females | 66.8 ± 3.2 | 67.4 ± 3.3 | 67.0 ± 3.5 |
| Males | 77.2 ± 7.2 | 77.4 ± 7.2 | 77.7 ± 6.9 |

TABLE 2
Study 1 Area Under the Curve, Concentration peak (Cₚₑₐₖ) and
Time to Reach Concentration Peak (tₚₑₐₖ).

| | Study 1 Treatment¹ | | | |
|---|---|---|---|---|
| | | Free Acid Gel | Free Acid Gel | p |
| | CaHMB | Swallow | Sublingual | Value² |
| Cₚₑₐₖ, nmol/mL | 131.2 ± 10.1 | 259.1 ± 23.9 | 231.2 ± 21.0 | 0.0001 |
| tₚₑₐₖ, min | 121.9 ± 15.6 | 33.1 ± 4.6 | 36.3 ± 1.3 | 0.0001 |
| AUC, nmol/ | 13,997 ± 1,534 | 27,532 ± 1,742 | 26,778 ± 1,980 | 0.0001 |
| 180 min | | | | |

¹Mean ± SEM.
²P value for treatment differences.

TABLE 3
Blood Chemistry Study1.¹

| | CaHMB | | | Free Acid Gel Swallow | | |
|---|---|---|---|---|---|---|
| | | | % | | | % |
| | Before | After | Change | | After | Change |
| Glucose, mg/dL | 72.6 ± 1.6 | 76.4 ± 3.2 ± | 5.2% | 70.5 ± 1.7 | 73.3 ± 4.1 | 3.9% |
| Uric Acid, mg/dL | 4.5 ± 0.3 | 4.5 ± 0.4 | −1.4% | 4.9 ± 0.4 | 4.9 ± 0.4 | 0.8% |
| Blood Urea Nitrogen, mg/dL | 12.1 ± 1.0 | 11.3 ± 0.9 | −7.2% | 15.4 ± 1.6 | 14.4 ± 1.5 | −6.5% |
| Creatinine, mg/dL | 0.9 ± 0.1 | 0.9 ± 0.1 | 0.5% | 1.0 ± 0.1 | 0.9 ± 0.1 | −4.7% |
| Sodium, mEq/L | 139.1 ± 0.5 | 139.4 ± 0.8 | 0.2% | 138.5 ± 1.1 | 138.3 ± 0.9 | −0.2% |
| Potassium, mEq/L | 4.1 ± 0.1 | 4.7 ± 0.2 | 13.4% | 4.2 ± 0.1 | 4.6 ± 0.3 | 11.4% |
| Chloride, mEq/L | 103.3 ± 0.6 | 103.1 ± 0.7 | −0.1% | 103.6 ± 0.8 | 103.3 ± 0.5 | −0.4% |
| CO₂, mEq/L | 23.4 ± 0.6 | 24.8 ± 0.5 | 5.9% | 22.4 ± 0.7 | 24.3 ± 0.5 | 8.1% |
| Phosphorus, mg/dL | 3.9 ± 0.2 | 3.5 ± 0.3 | −10.8% | 4.0 ± 0.1 | 3.4 ± 0.1 | −15.6% |
| Protein, g/dL | 6.3 ± 0.2 | 6.5 ± 0.3 | 3.2% | 6.2 ± 0.2 | 6.3 ± 0.2 | 1.4% |
| Albumin, g/dL | 4.0 ± 0.1 | 4.1 ± 0.1 | 0.3% | 4.0 ± 0.2 | 4.1 ± 0.2 | 1.9% |
| Globulin, g/dL | 2.3 ± 0.2 | 2.5 ± 0.2 | 8.2% | 2.2 ± 0.1 | 2.2 ± 0.1 | 0.6% |
| A:G Ratio² | 1.8 ± 0.1 | 1.7 ± 0.1 | −8.9% | 1.9 ± 0.1 | 1.9 ± 0.1 | 0.0% |
| Total Bilirubin, mg/dL | 0.5 ± 0.07 | 0.5 ± 0.08 | 7.9% | 0.4 ± 0.05 | 0.5 ± 0.07 | 23.3% |
| Direct Bilirubin, mg/dL | 0.1 ± 0.02 | 0.1 ± 0.01 | 5.8% | 0.1 ± 0.01 | 0.1 ± 0.01 | 7.9% |
| Alkaline Phosphatase, IU/L | 66.5 ± 6.5 | 68.4 ± 8.4 | 2.8% | 63.9 ± 5.8 | 64.4 ± 6.7 | 0.8% |
| Lactate Dehydrogenase, IU/L | 160.9 ± 9.5 | 165.6 ± 10.7 | 3.0% | 168.8 ± 10.4 | 166.3 ± 11.1 | −1.5% |
| Aspartate Aminotransferase, IU/L | 21.4 ± 1.5 | 22.4 ± 1.8 | 4.7% | 24.0 ± 2.2 | 23.1 ± 2.0 | −3.6% |
| Alanine Aminotransferase, IU/L | 14.1 ± 1.5 | 15.6 ± 2.0 | 10.6% | 15.9 ± 1.5 | 15.6 ± 1.3 | −1.6% |
| Gamma-glutamyl | 13.8 ± 0.9 | 13.1 ± 0.9 | −4.5% | 14.4 ± 1.6 | 13.5 ± 1.1 | −6.1% |
| Iron Binding Capacity, μg/dL | 330.4 ± 32.9 | 342.8 ± 35.9 | 3.7% | 328.6 ± 31.0 | 333.4 ± 35.2 | 1.4% |
| UIBC, μg/dL | 247.1 ± 45.0 | 249.8 ± 47.7 | 1.1% | 243.1 ± 47.4 | 237.1 ± 49.2 | −2.5% |
| Iron, μg/dL | 83.3 ± 16.7 | 93.0 ± 16.8 | 11.7% | 85.5 ± 19.2 | 96.3 ± 17.3 | 12.6% |
| Iron Saturation, % | 29.9 ± 9.5 | 31.1 ± 8.9 | 4.2% | 30.6 ± 9.4 | 33.5 ± 8.4 | 9.4% |
| Total Cholesterol, mg/dL | 153.4 ± 9.1 | 153.9 ± 9.7 | 0.3% | 153.5 ± 5.3 | 153.3 ± 5.6 | −0.2% |
| Triglycerides, mg/dL | 80.4 ± 11.6 | 71.6 ± 11.2 | −10.9% | 91.3 ± 17.0 | 87.5 ± 14.8 | −4.1% |
| HDL, mg/dL | 51.3 ± 3.2 | 53.1 ± 3.8 | 3.7% | 46.9 ± 3.4 | 48.5 ± 3.7 | 3.5% |
| LDL, mg/dL | 86.1 ± 5.3 | 86.5 ± 5.7 | 0.4% | 88.4 ± 3.0 | 85.6 ± 4.1 | −3.1% |
| Cholesterol Ratio | 3.0 ± 0.1 | 3.0 ± 0.1 | −2.5% | 3.4 ± 0.2 | 3.3 ± 0.3 | −3.3% |

| | Free Acid Gel Sublingual | | | |
|---|---|---|---|---|
| | | | % | |
| | Before | After | Change | p Value² |
| Glucose, mg/dL | 74.1 ± 1.7 | 77.4 ± 2.5 | 4.4% | 0.83 |
| Uric Acid, mg/dL | 4.0 ± 0.4 | 4.0 ± 0.5 | −2.2% | 0.47 |
| Blood Urea Nitrogen, mg/dL | 14.4 ± 0.8 | 12.9 ± 0.8 | −10.4% | 0.25 |
| Creatinine, mg/dL | 1.0 ± 0.1 | 1.0 ± 0.04 | −1.5% | 0.41 |
| Sodium, mEq/L | 139.3 ± 0.8 | 137.8 ± 0.9 | −1.1% | 0.25 |
| Potassium, mEq/L | 4.0 ± 0.03 | 4.5 ± 0.1 | 10.6% | 0.98 |
| Chloride, mEq/L | 102.9 ± 0.6 | 102.4 ± 1.1 | −0.5% | 0.98 |
| CO₂, mEq/L | 24.0 ± 0.5 | 24.3 ± 0.6 | 1.0% | 0.17 |
| Phosphorus, mg/dL | 4.1 ± 0.05 | 3.5 ± 0.2 | −13.2% | 0.71 |
| Protein, g/dL | 6.3 ± 0.2 | 6.4 ± 0.2 | 1.6% | 0.77 |
| Albumin, g/dL | 4.1 ± 0.1 | 4.1 ± 0.2 | 1.5% | 0.77 |
| Globulin, g/dL | 2.3 ± 0.2 | 2.3 ± 0.1 | 1.6% | 0.40 |
| A:G Ratio² | 1.9 ± 0.1 | 1.8 ± 0.1 | −2.0% | 0.46 |
| Total Bilirubin, mg/dL | 0.4 ± 0.05 | 0.5 ± 0.05 | 22.6% | 0.27 |
| Direct Bilirubin, mg/dL | 0.1 ± 0.01 | 0.1 ± 0.01 | 8.6% | 0.96 |
| Alkaline Phosphatase, IU/L | 66.0 ± 6.3 | 66.8 ± 6.8 | 1.1% | 0.80 |
| Lactate Dehydrogenase, IU/L | 164.8 ± 12.5 | 167.3 ± 12.2 | 1.5% | 0.87 |
| Aspartate Aminotransferase, IU/L | 28.0 ± 7.9 | 28.3 ± 8.2 | 0.9% | 0.15 |
| Alanine Aminotransferase, IU/L | 17.4 ± 2.7 | 17.3 ± 2.7 | −0.7% | 0.25 |
| Gamma-glutamyl | 13.3 ± 1.2 | 13.4 ± 1.2 | 0.9% | 0.45 |
| Iron Binding Capacity, μg/dL | 328.0 ± 32.8 | 339.5 ± 30.8 | 3.5% | 0.79 |
| UIBC, μg/dL | 244.8 ± 42.3 | 244.9 ± 39.5 | 0.1% | 0.83 |
| Iron, μg/dL | 83.3 ± 16.4 | 94.6 ± 16.6 | 13.7% | 0.79 |
| Iron Saturation, % | 29.1 ± 9.0 | 30.9 ± 8.5 | 6.0% | 0.53 |
| Total Cholesterol, mg/dL | 154.4 ± 6.9 | 155.5 ± 5.8 | 0.7% | 0.90 |
| Triglycerides, mg/dL | 91.8 ± 16.1 | 83.8 ± 15.8 | −8.7% | 0.62 |
| HDL, mg/dL | 48.6 ± 4.6 | 49.3 ± 4.3 | 1.3% | 0.69 |
| LDL, mg/dL | 87.5 ± 5.7 | 89.4 ± 4.9 | 2.1% | 0.48 |
| Cholesterol Ratio | 3.3 ± 0.2 | 3.3 ± 0.2 | −0.4% | 0.72 |

¹Mean ± Standard Error of the Mean.
²P value for treatment effect of the difference in starting and ending values indicated by % change for each treatment.

TABLE 4
Hematology Values Study 1.¹

| | CaHMB | | | HMB-Acid Gel Swallow | | |
|---|---|---|---|---|---|---|
| | | | % | | | % |
| | Before | After | Change | Before | After | Change |
| WBC, ×10³/μL | 6.4 ± 0.4 | 6.4 ± 0.7 | 0.3% | 6.7 ± 0.5 | 6.1 ± 0.3 | −10.2% |
| RBC, ×10³/μL | 4.3 ± 0.2 | 4.3 ± 0.2 | 1.0% | 4.2 ± 0.2 | 4.2 ± 0.2 | 0.1% |
| Hemoglobin, g/dL | 13.3 ± 0.4 | 13.4 ± 0.5 | 1.2% | 13.1 ± 0.5 | 13.0 ± 0.5 | −0.4% |
| Hematocrit, % | 38.3 ± 1.1 | 38.7 ± 1.3 | 1.2% | 37.6 ± 1.6 | 37.6 ± 1.5 | −0.1% |
| MCV, fL | 90.0 ± 1.5 | 90.4 ± 1.5 | 0.4% | 90.1 ± 1.3 | 90.0 ± 1.5 | −0.1% |
| MCH, pg | 31.2 ± 0.6 | 31.3 ± 0.6 | 0.3% | 31.2 ± 0.7 | 31.4 ± 0.8 | 0.7% |
| MCHC, g/dL | 34.7 ± 0.1 | 34.7 ± 0.2 | 0.1% | 34.7 ± 0.3 | 34.6 ± 0.3 | −0.3% |
| RDW, % | 13.1 ± 0.2 | 13.1 ± 0.2 | 0.1% | 13.2 ± 0.2 | 13.2 ± 0.3 | 0.4% |
| Platelets, ×10³/μL | 209.0 ± 14 | 202.9 ± 16 | −2.9% | 195.0 ± 31.5 | 202.4 ± 25. | 3.8% |
| Neutrophils, % | 50.3 ± 3.3 | 56.1 ± 3.8 | 11.7% | 46.0 ± 2.8 | 52.9 ± 2.2 | 14.9% |
| Lymphocytes, % | 39.6 ± 2.8 | 34.0 ± 3.4 | −14.2% | 43.8 ± 2.8 | 37.4 ± 1.9 | −14.6% |
| Monocytes, % | 7.1 ± 0.9 | 7.4 ± 0.7 | 3.5% | 7.6 ± 0.7 | 7.0 ± 0.6 | −8.2% |
| Eosinophils, % | 2.5 ± 0.4 | 2.1 ± 0.6 | −15.0% | 2.5 ± 0.3 | 2.4 ± 0.4 | −5.0% |
| Basophils, % | 0.5 ± 0.2 | 0.4 ± 0.2 | −25.0% | 0.1 ± 0.1^{a} | 0.4 ± 0.2 | 200.0% |
| Neutrophils, ×10³/μL | 3.3 ± 0.4 | 3.8 ± 0.7 | 14.5% | 3.0 ± 0.2 | 3.3 ± 0.2 | 10.9% |
| Lymphocytes, ×10³/μL | 2.5 ± 0.1 | 2.1 ± 0.2 | −15.6% | 3.1 ± 0.3 | 2.2 ± 0.1 | −29.1% |
| Monocytes, ×10³/μL | 0.5 ± 0.1 | 0.5 ± 0.1 | 8.3% | 0.5 ± 0.1 | 0.5 ± 0.04 | −7.7% |
| Eosinophils, ×10³/μL | 0.2 ± 0.04 | 0.1 ± 0.04 | −21.4% | 0.2 ± 0.04 | 0.1 ± 0.02 | −41.2% |
| Basophils, ×10³/μL | 0.05 ± 0.0 | 0.04 ± 0.0 | −25.0% | 0.01 ± 0.01^{a} | 0.04 ± 0.02 | 200.0% |

| | HMB-Acid Gel Sublingual | | | |
|---|---|---|---|---|
| | | | % | |
| | Before | After | Change | p Value² |
| WBC, ×10³/μL | 6.6 ± 0.4 | 6.3 ± 0.4 | −4.2% | 0.39 |
| RBC, ×10³/μL | 4.2 ± 0.2 | 4.3 ± 0.2 | 1.7% | 0.51 |
| Hemoglobin, g/dL | 13.1 ± 0.6 | 13.3 ± 0.6 | 1.4% | 0.54 |
| Hematocrit, % | 37.8 ± 1.6 | 38.5 ± 1.7 | 2.0% | 0.44 |
| MCV, fL | 89.6 ± 1.6 | 89.9 ± 1.7 | 0.3% | 0.57 |
| MCH, pg | 31.0 ± 0.5 | 31.0 ± 0.5 | 0.0% | 0.76 |
| MCHC, g/dL | 34.7 ± 0.2 | 34.5 ± 0.2 | −0.5% | 0.64 |
| RDW, % | 13.2 ± 0.2 | 13.3 ± 0.2 | 0.9% | 0.73 |
| Platelets, ×10³/μL | 221.3 ± 20.8 | 213.9 ± 19.1 | −3.3% | 0.31 |
| Neutrophils, % | 47.4 ± 2.2 | 55.4 ± 2.9 | 16.9% | 0.78 |
| Lymphocytes, % | 43.0 ± 1.4 | 35.5 ± 2.1 | −17.4% | 0.55 |
| Monocytes, % | 7.0 ± 1.0 | 7.1 ± 0.7 | 1.8% | 0.85 |
| Eosinophils, % | 2.4 ± 0.4 | 1.8 ± 0.3 | −26.3% | 0.41 |
| Basophils, % | 0.3 ± 0.2 | 0.3 ± 0.2 | 0.0% | 0.14 |
| Neutrophils, ×10³/μL | 3.1 ± 0.3 | 3.5 ± 0.3 | 11.6% | 0.85 |
| Lymphocytes, ×10³/μL | 2.8 ± 0.2 | 2.2 ± 0.2 | −22.1% | 0.04 |
| Monocytes, ×10³/μL | 0.5 ± 0.1 | 0.5 ± 0.05 | 0.0% | 0.98 |
| Eosinophils, ×10³/μL | 0.2 ± 0.03 | 0.1 ± 0.02 | −28.6% | 0.47 |
| Basophils, ×10³/μL | 0.03 ± 0.02 | 0.03 ± 0.02 | 0.0% | 0.14 |

¹Mean ± Standard Error of the Mean.
²P value for treatment effect of the difference in starting and ending values indicated by % change for each treatment.

TABLE 5
Study 2 Plasma HMB Area Under the Curve, Concentration Peak (Cₚₑₐₖ),
Time to Concentration Peak (tₚₑₐₖ), Half-life, Urinary HMB Loss, and HMB Retention.

| | Study 2 Treatment¹ | | | |
|---|---|---|---|---|
| | | HMB-Acid Gel | HMB-Acid Gel | |
| | CaHMB | Swallow | Sublingual | p Value² |
| Cₚₑₐₖ, nmol/mL | 131.2 ± 6.0 | 238.6 ± 16.0 | 247.6 ± 19.8 | 0.0003 |
| tₚₑₐₖ, h | 135.0 ± 17.0 | 41.9 ± 5.8 | 38.8 ± 2.6 | 0.0001 |
| Half-life, h | 3.17 ± 0.22 | 2.50 ± 0.13 | 2.51 ± 0.14 | 0.004 |
| AUC, nmol/1440 | 46,281 ± 2,717 | 53,395 ± 2,862 | 52,886 ± 2,729 | 0.001 |
| min | | | | |
| 24 h Urine HMB, | 1.00 ± 0.13 | 1.21 ± 0.19 | 1.16 ± 0.17 | 0.18 |
| mmol | | | | |
| 24 h Urine HMB, | 14.7 ± 2.0 | 17.8 ± 2.9 | 17.2 ± 2.5 | 0.18 |
| % initial dose | | | | |
| Clearance | 53.9 ± 4.2 | 67.3 ± 3.2 | 66.9 ± 1.6 | 0.003 |
| (mL/min) | | | | |

¹Mean ± SEM.
²P value for treatment differences.
³At Cₚₑₐₖ total extracellular HMB was estimated using 20% body weight as the extracellular volume. It was assumed for this calculation the plasma and extracellular HMB concentrations were equalized. Amount retained was calculated by subtracting total amount of HMB excreted in the urine. Relative retention percentage indicated in the table should remain unchanged even if this assumption is not met.

TABLE 6
Blood Chemistry Study 2.¹

| | CaHMB | | | HMB-Acid Gel Swallow | | |
|---|---|---|---|---|---|---|
| | | | % | | | % |
| | Before | After | Change | Before | After | Change |
| Glucose, mg/dL | 70.8 ± 0.8 | 80.3 ± 2.4 | 13.4% | 74.1 ± 2.0 | 79.9 ± 1.9 | 7.8% |
| Uric Acid, mg/dL | 4.8 ± 0.3 | 4.2 ± 0.5 | −12.0% | 4.7 ± 0.1 | 4.8 ± 0.3 | 1.9% |
| Blood Urea Nitrogen, mg/dL | 14.8 ± 1.4 | 13.4 ± 1.0 | −9.3% | 14.5 ± 1.4 | 13.9 ± 1.4 | −4.3% |
| Creatinine, mg/dL | 1.0 ± 0.1 | 1.0 ± 0.1 | −1.5% | 1.0 ± 0.1 | 1.0 ± 0.1 | −0.6% |
| Sodium, mEq/L | 139.5 ± 0.8 | 141.4 ± 0.5 | 1.3% | 139.9 ± 0.7 | 141.5 ± 0.9 | 1.2% |
| Potassium, mEq/L | 4.1 ± 0.1 | 4.7 ± 0.1 | 14.8% | 4.1 ± 0.1 | 4.7 ± 0.2 | 14.0% |
| Chloride, mEq/L | 102.4 ± 0.5 | 103.0 ± 0.5 | 0.6% | 103.1 ± 0.8 | 102.8 ± 0.7 | −0.4% |
| CO₂, mEq/L | 24.9 ± 0.6 | 27.3 ± 0.6 | 9.5% | 24.8 ± 1.0 | 27.8 ± 0.9 | 12.1% |
| Phosphorus, mg/dL | 4.0 ± 0.1 | 4.4 ± 0.1 | 10.6% | 4.2 ± 0.2 | 4.5 ± 0.2 | 6.2% |
| Protein, g/dL | 6.4 ± 0.2 | 6.5 ± 0.2 | 2.8% | 6.4 ± 0.1 | 6.7 ± 0.2 | 5.1% |
| Albumin, g/dL | 4.1 ± 0.1 | 4.2 ± 0.1 | 0.6% | 4.1 ± 0.1 | 4.3 ± 0.1 | 4.5% |
| Globulin, g/dL | 2.2 ± 0.1 | 2.4 ± 0.1 | 6.7% | 2.3 ± 0.2 | 2.4 ± 0.2 | 6.0% |
| A:G Ratio² | 1.9 ± 0.1 | 1.8 ± 0.1 | −3.4% | 1.9 ± 0.2 | 1.8 ± 0.1 | −2.6% |
| Total Bilirubin, mg/dL | 0.6 ± 0.1 | 0.5 ± 0.2 | −17.8% | 0.5 ± 0.1 | 0.5 ± 0.1 | 2.6% |
| Direct Bilirubin, mg/dL | 0.2 ± 0.1 | 0.1 ± 0.04 | −37.4% | 0.1 ± 0.03 | 0.1 ± 0.04 | 6.0% |
| Alkaline Phosphatase, IU/L | 65.5 ± 7.3 | 66.8 ± 6.9 | 1.9% | 64.8 ± 7.3 | 74.1 ± 7.6 | 14.5% |
| Lactate Dehydrogenase, IU/L | 161.8 ± 12.6 | 142.4 ± 10.5 | −12.0% | 155.9 ± 13.9 | 136.6 ± 11.6 | −12.3% |
| Aspartate Aminotransferase, | 22.6 ± 1.4 | 20.0 ± 1.1 | −11.6% | 23.3 ± 0.9 | 21.3 ± 0.7 | −8.6% |
| Alanine Aminotransferase, IU/L | 15.4 ± 1.8 | 14.9 ± 2.2 | −3.3% | 16.4 ± 1.6 | 16.4 ± 1.7 | 0.0% |
| Gamma-glutamyl | 14.0 ± 1.9 | 14.3 ± 1.4 | 1.8% | 13.3 ± 1.2 | 14.0 ± 1.0 | 5.7% |
| Iron Binding Capacity, μg/dL | 371.8 ± 28.6 | 377.5 ± 30.3 | 1.5% | 378.6 ± 32.1 | 395.1 ± 34.7 | 4.4% |
| UIBC, μg/dL | 272.8 ± 39.5 | 317.6 ± 38.3 | 16.5% | 310.6 ± 42.4 | 335.1 ± 42.1 | 7.9% |
| Iron, μg/dL | 99.0 ± 20.0 | 59.9 ± 12.2 | −39.5% | 68.0 ± 13.4 | 53.8 ± 10.8 | −21.0% |
| Iron Saturation, % | 28.4 ± 5.8 | 17.5 ± 4.2 | −38.3% | 20.3 ± 5.0 | 16.9 ± 4.6 | −16.7% |
| Total Cholesterol, mg/dL | 160.4 ± 8.0 | 162.9 ± 7.1 | 1.6% | 164.6 ± 10.5 | 176.9 ± 11.8 | 7.4% |
| Triglycerides, mg/dL | 97.4 ± 12.5 | 106.9 ± 17.6 | 9.8% | 112.6 ± 18.4 | 121.4 ± 22.7 | 7.8% |
| HDL, mg/dL | 52.3 ± 2.7 | 54.0 ± 3.5 | 3.3% | 51.4 ± 2.8 | 56.4 ± 2.9 | 9.7% |
| LDL, mg/dL | 88.6 ± 7.2 | 88.6 ± 5.9 | 0.0% | 90.8 ± 7.0 | 96.1 ± 8.1 | 5.9% |
| Cholesterol Ratio | 3.1 ± 0.2 | 3.1 ± 0.2 | −0.8% | 3.2 ± 0.2 | 3.2 ± 0.2 | −2.3% |

| | HMB-Acid Gel Sublingual | | | |
|---|---|---|---|---|
| | | | % | |
| | Before | After | Change | p Value² |
| Glucose, mg/dL | 73.0 ± 1.8 | 80.8 ± 2.1 | 10.6% | 0.62 |
| Uric Acid, mg/dL | 4.7 ± 0.3 | 4.7 ± 0.3 | 0.8% | 0.50 |
| Blood Urea Nitrogen, mg/dL | 15.3 ± 1.7 | 15.4 ± 1.1 | 0.8% | 0.55 |
| Creatinine, mg/dL | 1.0 ± 0.1 | 1.0 ± 0.1 | 7.9% | 0.12 |
| Sodium, mEq/L | 138.5 ± 0.6 | 142.1 ± 1.1 | 2.6% | 0.07 |
| Potassium, mEq/L | 4.0 ± 0.1 | 4.9 ± 0.1 | 21.7% | 0.51 |
| Chloride, mEq/L | 102.1 ± 0.5 | 104.3 ± 0.9 | 2.1% | 0.06 |
| CO₂, mEq/L | 25.3 ± 0.8 | 27.9 ± 0.4 | 10.4% | 0.85 |
| Phosphorus, mg/dL | 4.1 ± 0.1 | 4.6 ± 0.2 | 11.5% | 0.44 |
| Protein, g/dL | 6.5 ± 0.2 | 6.6 ± 0.2 | 2.9% | 0.62 |
| Albumin, g/dL | 4.1 ± 0.1 | 4.2 ± 0.1 | 2.7% | 0.08 |
| Globulin, g/dL | 2.3 ± 0.1 | 2.4 ± 0.1 | 3.2% | 0.93 |
| A:G Ratio² | 1.8 ± 0.1 | 1.8 ± 0.1 | −1.4% | 0.83 |
| Total Bilirubin, mg/dL | 0.6 ± 0.1 | 0.5 ± 0.1 | −13.3% | 0.10 |
| Direct Bilirubin, mg/dL | 0.1 ± 0.03 | 0.1 ± 0.04 | −12.8% | 0.21 |
| Alkaline Phosphatase, IU/L | 66.5 ± 7.3 | 62.8 ± 6.7 | −5.6% | 0.18 |
| Lactate Dehydrogenase, IU/L | 163.8 ± 11.9 | 151.8 ± 5.2 | −7.3% | 0.86 |
| Aspartate Aminotransferase, | 23.9 ± 3.2 | 24.8 ± 2.3 | 3.7% | 0.23 |
| Alanine Aminotransferase, IU/L | 15.3 ± 1.5 | 16.6 ± 1.8 | 9.0% | 0.18 |
| Gamma-glutamyl | 13.9±1.2 | 13.9 ± 1.0 | 0.0% | 0.50 |
| Iron Binding Capacity, μg/dL | 371.9 ± 31.4 | 385.0 ± 29.8 | 3.5% | 0.68 |
| UIBC, μg/dL | 264.3 ± 33.3 | 312.0 ± 35.9 | 18.1% | 0.44 |
| Iron, μg/dL | 107.6 ± 13.7 | 65.0 ± 12.1 | −39.6% | 0.37 |
| Iron Saturation, % | 30.1 ± 5.1 | 20.1 ± 4.2 | −33.2% | 0.38 |
| Total Cholesterol, mg/dL | 161.6 ± 9.4 | 163.3 ± 9.3 | 1.0% | 0.25 |
| Triglycerides, mg/dL | 93.1 ± 10.6 | 107.6 ± 16.6 | 15.6% | 0.52 |
| HDL, mg/dL | 52.3 ± 3.1 | 51.3 ± 2.8 | −1.9% | 0.24 |
| LDL, mg/dL | 91.3 ± 5.9 | 90.4 ± 6.8 | −1.0% | 0.18 |
| Cholesterol Ratio | 3.1 ± 0.2 | 3.2 ± 0.2 | 3.6% | 0.53 |

¹Mean ± Standard Error of the Mean.
²P value for treatment effect of the difference in starting and ending values indicated by % change for each treatment.

TABLE 7
Hematology Values Study 2.¹

| | | | | HMB-Acid Gel Swallow | | |
|---|---|---|---|---|---|---|
| | CaHMB | | | | | % |
| | Before | After | % Change | Before | After | Change |
| WBC, ×10³/μL | 7.3 ± 0±.3 | 6.5 ± 0.3 | −10.5% | 6.4 ± 0.3 | 7.1 ± 0.3 | 9.7% |
| RBC, ×10³/μL | 4.3 ± 0.2 | 4.3 ± 0.2 | 0.7% | 4.2 ± 0.1 | 4.4 ± 0.1 | 3.5% |
| Hemoglobin, g/dL | 13.2 ± 0.7 | 13.3 ± 0.6 | 0.7% | 13.0 ± 0.4 | 13.4 ± 0.4 | 3.2% |
| Hematocrit, % | 38.1 ± 1.8 | 38.3 ± 1.7 | 0.7% | 37.4 ± 1.0 | 38.9 ± 1.1 | 4.2% |
| MCV, fL | 88.4 ± 1.5 | 88.5 ± 1.4 | 0.1% | 88.1 ± 1.5 | 88.6 ± 1.5 | 0.6% |
| MCH, pg | 30.6 ± 0.5 | 30.7 ± 0.6 | 0.2% | 30.6 ± 0.6 | 30.5 ± 0.6 | −0.3% |
| MCHC, g/dL | 34.7 ± 0.2 | 34.7 ± 0.2 | 0.0% | 34.8 ± 0.2 | 34.5 ± 0.2 | −0.8% |
| RDW, % | 13.4 ± 0.2 | 13.4 ± 0.1 | −0.1% | 13.3 ± 0.2 | 13.2 ± 0.2 | −0.5% |
| Platelets, ×10³/μL | 228.0 ± 17.6 | 235.9 ± 10.9 | 3.5% | 229.0 ± 12.9 | 242.4 ± 12.2 | 5.8% |
| Neutrophils, % | 44.9 ± 3.8 | 45.6 ± 3.3 | 1.7% | 45.6 ± 2.7 | 46.0 ± 4.4 | 0.8% |
| Lymphocytes, % | 44.8 ± 3.2 | 42.5 ± 3.5 | −5.0% | 43.6 ± 2.4 | 43.3 ± 3.9 | −0.9% |
| Monocytes, % | 6.9 ± 0.9 | 8.5 ± 1.5 | 23.6% | 7.5 ± 1.1 | 6.9 ± 0.8 | −8.3% |
| Eosinophils, % | 2.9 ± 0.5 | 2.6 ± 0.4 | −8.7% | 2.8 ± 0.5 | 3.0 ± 0.5 | 9.1% |
| Basophils, % | 0.6 ± 0.2 | 0.8 ± 0.2 | 20.0% | 0.5 ± 0.2 | 0.9 ± 0.3 | 75.0% |
| Neutrophils, ×10³/μL | 3.3 ± 0.3 | 3.0 ± 0.2 | −9.9% | 2.9 ± 0.2 | 3.3 ± 0.4 | 10.6% |
| Lymphocytes, ×10³/μL | 3.3 ± 0.3 | 2.8 ± 0.3 | −13.5% | 2.8 ± 0.2 | 3.1 ± 0.3 | 9.4% |
| Monocytes, ×10³/μL | 0.5 ± 0.1 | 0.5 ± 0.1 | 5.0% | 0.5 ± 0.1 | 0.5 ± 0.04 | 2.6% |
| Eosinophils, ×10³/μL | 0.2 ± 0.04 | 0.2 ± 0.03 | −12.5% | 0.2 ± 0.03 | 0.2 ± 0.04 | 13.3% |
| Basophils, ×10³/μL | 0.06 ± 0.02 | 0.06 ± 0.02 | 0.0% | 0.05 ± 0.02 | 0.09 ± 0.03 | 75.0% |

| | HMB-Acid Gel Sublingual | | | |
|---|---|---|---|---|
| | Before | After | % Change | p Value² |
| WBC, ×10³/μL | 7.1 ± 0.4 | 6.8 ± 0.3 | −3.5% | 0.10 |
| RBC, ×10³/μL | 4.3 ± 0.1 | 4.3 ± 0.1 | 0.1% | 0.27 |
| Hemoglobin, g/dL | 13.2 ± 0.5 | 13.2 ± 0.5 | 0.4% | 0.31 |
| Hematocrit, % | 37.5 ± 1.4 | 38.6 ± 1.3 | 2.9% | 0.42 |
| MCV, fL | 88.4 ± 1.5 | 89.3 ± 1.4 | 1.0% | 0.21 |
| MCH, pg | 30.4 ± 0.5 | 30.5 ± 0.5 | 0.3% | 0.52 |
| MCHC, g/dL | 34.5 ± 0.2 | 34.5 ± 0.2 | 0.1% | 0.14 |
| RDW, % | 13.3 ± 0.2 | 13.4 ± 0.2 | 0.7% | 0.66 |
| Platelets, ×10³/μL | 233.8 ± 14.2 | 244.3 ± 17.5 | 4.5% | 0.92 |
| Neutrophils, % | 45.3 ± 5.2 | 42.4 ± 3.5 | −6.4% | 0.90 |
| Lymphocytes, % | 43.1 ± 4.2 | 45.6 ± 3.4 | 5.8% | 0.68 |
| Monocytes, % | 8.3 ± 1.0 | 8.4 ± 0.9 | 1.5% | 0.24 |
| Eosinophils, % | 2.5 ± 0.5 | 3.3 ± 0.5 | 30.0% | 0.57 |
| Basophils, % | 0.6 ± 0.2 | 0.4 ± 0.2 | −40.0% | 0.36 |
| Neutrophils, ×10³/μL | 3.3 ± 0.5 | 2.9 ± 0.3 | −12.1% | 0.49 |
| Lymphocytes, ×10³/μL | 3.0 ± 0.3 | 3.1 ± 0.3 | 5.0% | 0.06 |
| Monocytes, ×10³/μL | 0.6 ± 0.05 | 0.6 ± 0.05 | 0.0% | 0.71 |
| Eosinophils, ×10³/μL | 0.2 ± 0.04 | 0.2 ± 0.03 | 35.7% | 0.42 |
| Basophils, ×10³/μL | 0.06 ± 0.02 | 0.04 ± 0.0 | −40.0% | 0.29 |

¹Mean ± Standard Error of the Mean.
²P value for treatment effect of the difference in starting and ending values indicated by % change for each treatment.

### Example 2

In this example the effect of administration of HMB-acid gel is compared to that of calcium HMB on muscle damage after an eccentric bout of exercise. As shown in Example 1, peak plasma HMB levels and HMB clearance rate are increased with HMB free acid gel administration compared with CaHMB (13). This example shows that the quicker response of HMB administered as free acid gel prior to and following a bout of extreme exercise protects the muscle from damage better than HMB administered as the calcium HMB salt.

Effects of HMB and exercise on markers of muscle damage and inflammatory factors: Strenuous exercise, such as resistance training or maximal effort exercise, causes an increase in leakage of the enzyme creatine phosphokinase (CPK) from muscle cells (21; 31). Human studies have shown that muscle damage following intense exercise, measured by elevated plasma CPK is reduced with calcium HMB supplementation (14; 22; 26; 33). A study on muscle damage after a prolonged 20 km run on a collegiate cross country course with both inclines and declines also showed chronic calcium HMB administration is effective in decreasing the rise in plasma CPK over a 4 day period following the run (23). Nissen et al. (26) demonstrated a dosage effect of calcium HMB (with 3.0 gram per day being more effective than 1.5 grams per day) in decreasing CPK as well as resulting in significant reductions in urinary 3-methylhistidine (3-MH, a well established indicator of myofibrillar protein degradation (39)). Gallagher et al. supplemented dosages of HMB to 37 male college students, performing resistance exercise training, corresponding to 3.0 (38 mg/kg body weight-d⁻¹) and 6.0 g (76 mg/kg body weight-d⁻¹) of calcium HMB per day. Both doses had similar effects on improving lean mass and strength gains, however, the higher dosage resulted in significant improvements in minimizing CPK leakage suggesting significant amelioration in muscle damage following exercise. These observations indicate that higher plasma levels of HMB appear to more protective of muscle damage following exercise.

As shown in Example 1, HMB as a free acid (in a gel) is absorbed faster than calcium HMB, results in higher plasma levels of HMB, and is cleared more readily by muscle.

### Methods:

Subjects: We conducted a study at the Iowa State University Health and Human Performance Laboratory; this study was approved by the Iowa State University Institutional Review Board, and was registered at ClinicalTrials.gov (NCT01150526). We recruited 12 men and 13 women between the ages of 20 and 36 from the Iowa State University community and surrounding area.

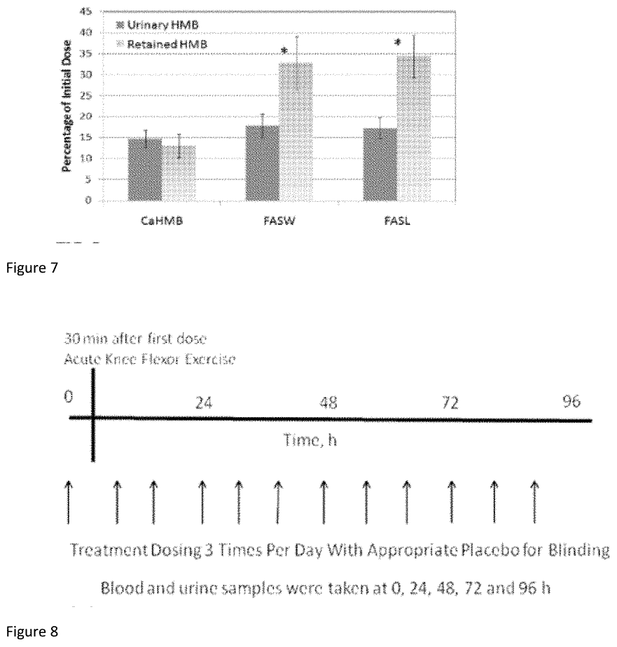

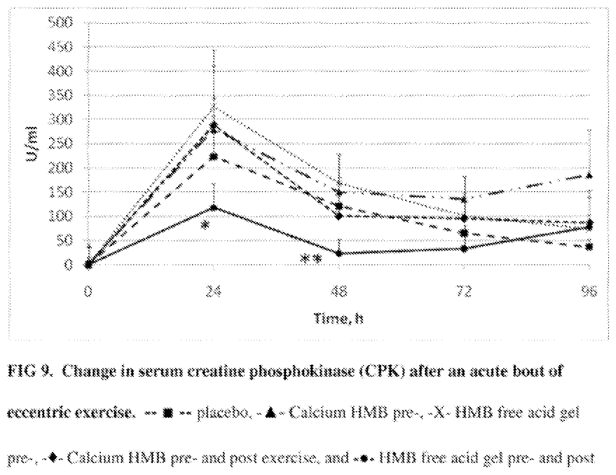

Treatments: The experimental design is depicted in FIG. 8.

Five treatments were administered as follows:
Treatment 1: Placebo. This group received a placebo capsule and a placebo syringe dosage at each dosage administration.
Treatment 2: CaHMB pre-exercise. This group received a CaHMB capsule and a placebo syringe dosage 30 min prior to the acute exercise bout. The remaining additional dosages during the study consisted of one placebo capsule and one placebo syringe dosage.
Treatment 3: HMB-acid gel pre-exercise. This group received a placebo capsule and a syringe dosage of HMB-acid gel 30 min prior to the acute exercise bout. The remaining additional dosages during the study consisted of one placebo capsule and one placebo syringe dosage.
Treatment 4: CaHMB pre- and post-exercise. This group received a CaHMB capsule and a placebo syringe dosage at all administration times during the study.
Treatment 5: HMB-acid gel pre- and post-exercise. This group received a syringe dosage of HMB-acid gel and one placebo capsule at all administration times during the study.

To blind the treatments to both the researchers and subjects, each subject took a capsule and syringe dosage of supplement at each administration. The capsules contained either one gram of calcium lactate (Placebo) or one gram of calcium β-hydroxy-β-methylbutryate (CaHMB). The syringe dosages were formulated to be similar in taste and appearance and contained either 0.8 g of corn syrup (Placebo) or 0.8 g of β-hydroxy-β-methylbutryate free acid, the same amount of HMB as in the capsule dosage. The 3 daily doses provided the same total HMB dosage (2.4 g as calcium HMB in the capsules or 2.4 g as HMB free acid in the syringes).

Eccentric Exercise Bout: Subjects refrained from vigorous exercise for three days before reporting to the laboratory. All subjects were studied after an overnight fast. Subjects had a fasting blood sample taken and a spot urine sample was collected. Subjects then consumed their assigned supplement and 30 minutes later the eccentric exercise session was performed. The exercise consisted of 50 maximal effort contractions of knee extensors while attempting to resist the Biodex lever arm as it moves the knee joint from full extension to 90 degrees flexion; this is similar to letting a heavy weight down slowly while in a seated position. Each contraction lasted about 2 seconds and 12 seconds were allowed between contractions. This protocol was performed on the right leg followed by the left leg. The subjects received 2 more daily dosages of treatment (placebo or HMB) with instructions to take the dosages at lunch and dinner. For the next 4 days the subjects returned to the laboratory fasted each morning, had blood taken and urine collected and then consumed the morning dosage of their supplement. The subjects were again given the 2 remaining daily supplement dosages with instructions to take them at lunch and dinner.

Serum and Urine Samples: A fasting blood sample was taken from a superficial forearm vein each morning when the subjects reported to the laboratory for testing. Additionally a clean catch urine sample was collected at this time. Serum CPK was analyzed by a commercial laboratory (Quest Diagnostics, Madison N.J.). Urine 3-Methylhistidine (3MH) was analyzed by a previously published GC/MS method (9). Urinary creatinine was analyzed by colorimetric assay (Cayman Chemical Company, Ann Arbor, Mich.). The urinary 3MH data was normalized to urinary creatinine and expressed as 3MH:creatinine ratio, μmol:mg.

Statistics: Data were analyzed using the mixed model procedure in Statistical Analysis System for Windows (Release 9.1.3, SAS Institute, Cary, N.C.). The change in each variable was analyzed at each measurement time point. The model included the baseline or time 0 value as a covariate and included gender and treatment as main effects. CPK data were transformed using the Rank procedure in SAS before analysis. Contrasts were used to compare the HMB-acid gel pre- and post exercise mean to the other treatment means.

Results:

The subjects' demographics are shown in Table 8. The age, height, and weight of the subjects by treatment were similar.

TABLE 8
Subject Characteristics

| | Treatment 1 | Treatment 2 | Treatment 3 | Treatment 4 | Treatment 5 |
|---|---|---|---|---|---|
| N | 5 | 4 | 5 | 5 | 6 |
| M/F | 3/2 | 2/2 | 2/3 | 2/3 | 3/3 |
| Age (y) | 25.2 ± 2.5 | 21.0 ± 0.4 | 24.0 ± 1.4 | 25.8 ± 2.6 | 22.2 ± 1.0 |
| Height (cm) | 173 ± 2.7 | 173 ± 5.5 | 170 ± 6.0 | 173 ± 6.0 | 171 ± 3.1 |
| Weight (kg) | 67.0 ± 2.3 | 68.9 ± 9.8 | 68.1 ± 5.8 | 69.7 ± 6.9 | 65.2 ± 5.1 |

^{a}Data are presented as Mean ± SEM.

Treatments were: (Treatment 1): placebo capsule and placebo syringe dosage; (Treatment 2) Calcium HMB capsule and placebo syringe dosage pre-exercise followed by placebo capsule and placebo syringe dosage post exercise; (Treatment 3): Placebo capsule and HMB-acid gel syringe dosage given pre-exercise followed by placebo capsule and placebo syringe dosage post exercise; (Treatment 4): Calcium HMB capsule and placebo syringe dosage pre- and post exercise; and (Treatment 5): Placebo capsule and HMB-acid gel syringe dosage given pre- and post exercise. Treatments were administered 3 times daily, 30 min before the morning testing and then again at approximately noon and 6 PM.

Serum CPK and urinary 3MH:urinary creatinine ratios are shown in Table 9. There were no differences in baseline (time 0) values. The eccentric exercise caused up to a four-fold increase in serum CPK, indicating muscle membrane damage. However, HMB-acid gel administered both pre- and post exercise (Treatment 5), blunted this increase by up to 64% (P<0.03) at 24 h post exercise and 86% 48 h post exercise (P<0.005). Continuing at 72 h the increase in CPK still tended to be less with the HMB-acid gel treatment. FIG. 9 illustrates the rise and fall in CPK values over the course of the study. Also shown in Table 2 is the effect of the treatments on 3MH:Cr ratio, a measure of protein degradation in muscle. Although these data only are indicative of a trend, the data do show no increase and even a decrease in protein degradation in the HMB-acid gel pre- and post exercise treatment. This would indicate a decrease in protein degradation resulting in more favorable protein turnover and protein accretion in muscle.

TABLE 9
Serum Creatine Phosphokinase (CPK) and Urinary 3-Methylhistidine to
Urinary Creatinine Ratio (3MH:Cr) After an Acute Bout of Exercise

| | | | | | |
|---|---|---|---|---|---|
| Treatment 1 | Treatment 2 | Treatment 3 | Treatment 4 | Treatment 5 | P-value^{b} |

CPK, U/mL

| | | | | | | |
|---|---|---|---|---|---|---|
| Baseline | 132 ± 35 | 95 ± 12 | 107 ± 17 | 127 ± 38 | 146 ± 43 | |
| at 24 h | 356 ± 108 | 375 ± 68 | 434 ± 126 | 416 ± 157 | 264 ± 75 | 0.03 |
| at 48 h | 253 ± 73 | 246 ± 38 | 275 ± 69 | 228 ± 73 | 170 ± 41 | 0.005 |
| at 72 h | 197 ± 48 | 231 ± 59 | 209 ± 54 | 223 ± 52 | 180 ± 34 | 0.09 |
| at 96 h | 169 ± 36 | 281 ± 105 | 181 ± 33 | 214 ± 52 | 187 ± 46 | 0.51 |

3MH:Cr, μmol/mg

| | | | | | | |
|---|---|---|---|---|---|---|
| Baseline | 0.233 ± 0.066 | 0.164 ± 0.037 | 0.173 ± 0.028 | 0.205 ± 0.025 | 0.225 ± 0.034 | |
| at 24 h | 0.211 ± 0.035 | 0.190 ± 0.027 | 0.213 ± 0.037 | 0.203 ± 0.039 | 0.213 ± 0.025 | 0.88 |
| at 48 h | 0.238 ± 0.033 | 0.240 ± 0.052 | 0.190 ± 0.014 | 0.220 ± 0.027 | 0.208 ± 0.012 | 0.62 |
| at 72 h | 0.251 ± 0.046 | 0.204 ± 0.058 | 0.172 ± 0.013 | 0.231 ± 0.037 | 0.175 ± 0.013 | 0.14 |
| at 96 h | 0.231 ± 0.044 | 0.187 ± 0.029 | 0.202 ± 0.020 | 0.269 ± 0.038 | 0.186 ± 0.043 | 0.27 |

^{a}Data are presented as Mean ± SEM.
Treatments were: (Treatment 1): placebo capsule and placebo syringe dosage; (Treatment 2) Calcium HMB capsule and placebo syringe dosage pre-exercise followed by placebo capsule and placebo syringe dosage post exercise; (Treatment 3): Placebo capsule and HMB free acid gel syringe dosage given pre-exercise followed by placebo capsule and placebo syringe dosage post exercise; (Treatment 4): Calcium HMB capsule and placebo syringe dosage pre- and post exercise; and (Treatment 5): Placebo capsule and HMB free acid gel syringe dosage given pre- and post exercise. Treatments were administered 3 times daily, 30 min before the morning testing and then again at approximately noon and 6 PM.
^{b}P-value HMB Free acid gel (Treatment 5) contrasted with the other treatments.

### Implications:

Based upon the observations in Example 1, muscle tissue was exposed to much higher levels of serum HMB when HMB free acid was administered. Additionally, in Example 1 it was shown that clearance of HMB from serum to muscle and tissues was also much greater when HMB-acid was administered compared with CaHMB administration. Thus, in Example 2 it is shown that this additional utilization of HMB by muscle in the free acid form is more protective of the muscle tissue after an acute bout of exercise than HMB administered in the calcium form.

### Example 3

A study was conducted to examine the bioavailability of HMB-acid and CaHMB when also administering a probiotic. Six volunteers were tested before and after two weeks of probiotic supplementation. Between trials, each volunteer took one dose of probiotics (BC³⁰) every morning for 14 days. At each trial, baseline plasma HMB levels were measured, then gave each volunteer one serving of HMB (1.0 g of CaHMB or 0.8 g of HMB-FA). Plasma HMB levels were measured at 2, 5, 10, 15, 25, 35, 45, 60, 90, 120, 150, and 180 min, as well as at 6, 12, and 24 hours.

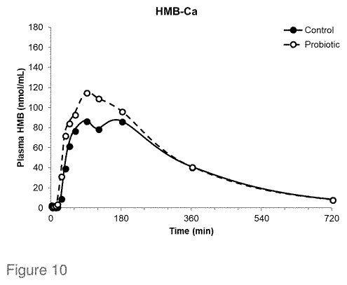

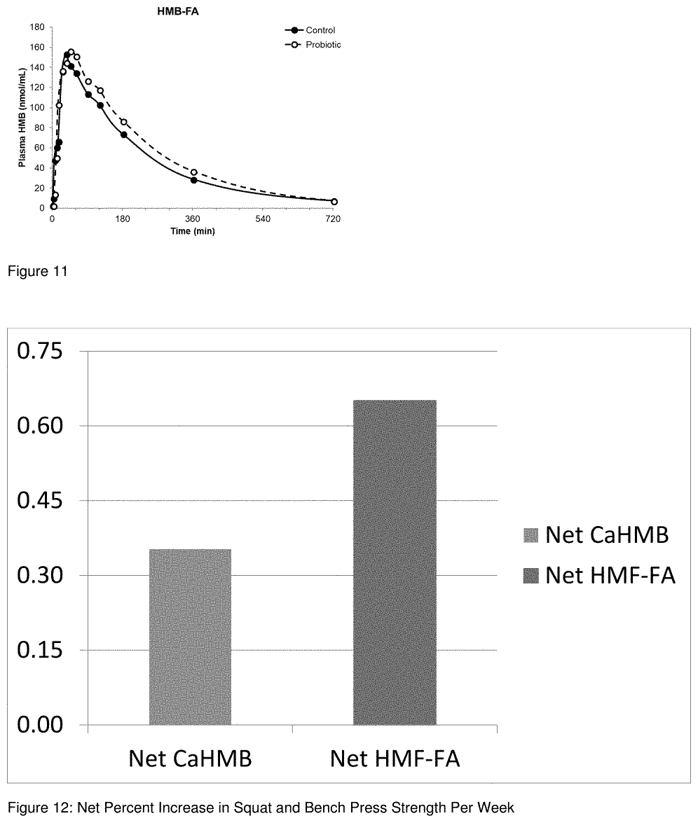

FIGS. 10 and 11 depict the peak HMB levels with the two treatments, demonstrating a faster time to peak plasma levels, greater retention of HMB, and improved bioavailability of HMB-acid. Higher bioavailability and optimized tissue exposure with HMB-acid results in greater effectiveness of HMB.

### Example 4

A study was conducted to examine the effects of CaHMB on strength. A second study was conducted to examine the effects of HMB-acid on strength. The results of these studies were then compared to determine the improved efficacy of HMB-acid over CaHMB on increasing strength gains.

Study 1—Effects of CaHMB on Strength

Thirty-two male volunteers 19-22 yr of age were selected for the study. Body weight averaged 99.3±3.4 kg with a range of 72-136 kg and height was 185±1.5 cm with a range of 170-198 cm. Almost all subjects were engaged in some form of exercise program before the study.

Body composition was measured with TOBEC except that all subjects were measured after an overnight fast. Measurements were made on the Friday morning before the start of the experiment and each Friday thereafter. Strength measurements were made the week before the study and consisted of three standard strength measurements: the bench press, the squat, and the hang clean. Treatments and exercise regimens were started on Monday. The exercise regimen consisted of weight training 6 day s/wk, which included work on all major muscle groups, and lasted from 2 to 3 h/day. Aerobic training was also included in the exercise workout at least three times per week.

No dietary control was imposed and the subjects were instructed to eat normally. Most meals were obtained at the Iowa State University athletic-training table. In addition, a nutrient shake was available during each training session. This was available to all subjects regardless of treatment and was served in the weight-training area.

The subjects were randomly assigned to one of two supplements, one of which contained CaHMB, so they did not know whether they were receiving CaHMB. Because we found no effect of added nutrient supplementation on HMB effects in study 1, CaHMB was delivered in the nutrient shake (MET-Rx). The placebo was an orange drink mix that contained calories equal to the nutrient shake but no added protein. Thus the two groups likely had different protein intakes, although we roughly estimated the total protein intake of the placebo group to be ˜180 g/day and the HMB group to be ˜200 g/day.

Differences between the two treatment groups were determined with at-test. Differences were considered significant if P<0.05. Trends were determined for 0.05<P<0.11, and differences were considered not significant for P>0.11.

Study 2—Effects of HMB-Acid on Strength

The current study was a randomized, double-blind, placebo- and diet-controlled experiment consisting of a 12-week-periodized resistance-training program. The training protocol was divided into three phases and consisted of a non-linear-periodized resistance-training program for the first 8 weeks, followed by a 2-week overreaching cycle, and finally a 2-week taper of the training volume. Muscle mass, body composition, strength, power, resting plasma testosterone, cortisol, and creatine kinase (CK) levels were examined collectively at the end of weeks 0, 4, 8, and 12 to assess the chronic effects of HMB; these measures were also assessed at the end of weeks 9 and 10, corresponding to the mid- and endpoints of the phase 2 overreaching cycle.

Twenty-four resistance-trained males were tested and included in the study. Subjects were organized into quartile blocks based on their LBM and strength. Following this, each quartile was randomized to one of the treatment groups using computer-generated random numbers. After treatment assignment, the groups were assessed to confirm there were no differences between groups. Of those randomized to the treatments, three subjects dropped from the placebo group, two because of injury and one because of the time commitment, while one dropped from the HMB-FA group due to injury. All drop outs occurred during the first 4 weeks of the study. The remaining 20 subjects (21.6±0.5 years of age) consisting of nine placebo (87.1±4.8 kg; 180.9 cm) and 11 supplemented (83.1±2.8 kg; 179.0±2.1) males with an average squat, bench press, and deadlift of 1.7±0.04, 1.3±0.04, and 2.0±0.05 times their bodyweight, respectively, completed the study. There were no significant differences for age, body weight, height, or BMI between the treatment groups at the start of the study

Strength was assessed via a one-repetition maximum (1-RM) testing of the back squat, bench press, and deadlift. The intraclass correlation coefficient (ICC) for the test-retest of strength in the squat, bench press, and deadlift ranged from r=0.956 to 0.982. Body composition [LBM, fat mass (FM), and total mass] was determined on a Lunar Prodigy dual X-ray absorptiometry (DXA) apparatus (software version, enCORE 2008, Madison, Wis., USA). Tests for the DXA were performed at the same time of day in a fasted state and the ICC was r=0.981. Skeletal muscle hypertrophy was assessed via changes in ultrasonography (GE Logiq e 2008, Wauwatosa, Wis., USA) determined combined muscle thickness of the vastus lateralis (VL) and vastus intermedius (VI) muscles. The ICC for the test-retest of muscle thickness measurements was r=0.975.

Peak power (PP) was assessed during maximal cycling (modified Wingate test) and jumping movements. During the cycling test, the volunteer was instructed to cycle against a predetermined resistance (7.5% of body weight) as fast as possible for 10 s on a cycle ergometer (Monark model 894e, Vansbro, Sweden). Wingate PP was recorded using Monark anaerobic test software (Version 1.0, Monark, Vansbro, Sweden). From completion of Wingate tests performed over several days, the ICC for Wingate PP was 0.966.

Measurements of PP for the vertical jump were also taken on a multicomponent AMTI force platform (Advanced Mechanical Technology, Watertown, Mass. USA) which interfaced with a personal computer at a sampling rate of 1,000 Hz). Data acquisition software (LabVIEW, version 7.1; National Instruments Corporation, Austin, Tex., USA) was used to calculate vertical jump PP. The ICC for VJ PP was 0.971.

Prior to the study, participants were randomly assigned to receive either 3 g per day of HMB-FA or a placebo divided equally into three servings. Each serving was formulated with 1 g of HMB-FA. The first serving was given 30 min prior to exercise and the remaining two servings given with the mid-day and evening meals. On the non-training days, participants were instructed to consume one serving with each of three separate meals throughout the day. Blinding occurred via an outside researcher who sent an isocaloric supplement and placebo in identical looking and flavored packets containing either 3 g per day of HMB-FA (combined with food-grade orange flavors and sweeteners) or a placebo (corn syrup combined with food-grade orange flavors and sweeteners) divided equally into three servings daily. The supplementation was continued daily throughout the training and testing protocols. Two weeks prior to and throughout the study, participants were placed on a diet consisting of 25% protein, 50% carbohydrates, and 25% fat by a registered dietician (M.S., RD, LD) who specialized in sports nutrition. Compliance was over 98% for supplementation.

The purity of HMB-FA was determined by the manufacturer (TSI, Missoula, Mont., USA) using high-pressure liquid chromatography to be 99.7%. The primary impurities were acetate and water. Metabolic Technologies Inc. (MTI, Ames, Iowa, USA) independently assayed the HMB-FA for purity and confirmed these results. In addition, MTI assayed HMB-FA for dehydroepiandrosterone (DHEA) a contaminant which has been found in nutritional supplements using gas chromatography-mass spectrometry (Thuyne and Delbeke 2005). DHEA was not detected in the HMB-FA (<1 ng/g). In addition, a sample was sent to an independent laboratory (MVTL, New Ulm, MN, USA) for microbial and heavy metals testing. HMB-FA tested negative for E. coli, Listeria, and Salmonella. Copper, zinc, calcium, mercury, cadmium, and lead were less than the instrument's detection limits and arsenic was detected at 37 PPB.

HMB-FA supplementation resulted in a significant increase in strength gain compared with placebo supplementation for squat, bench press, deadlift, and total strength). After 12 weeks of training, HMB-FA supplementation resulted in strength increases of 25% for the squat, 12% for the bench press, 16% for the deadlift, and 18% for total strength, which were significantly greater than the increases of 5% for the squat, 3% for the bench press, 9% for the deadlift, and 6% for total strength in the placebo group. Total strength increases over the 12-week study were 25.3±22.0 kg in the placebo-supplemented participants, and 77.1±18.4 kg in the HMB-FA-supplemented participants. Mean total strength also demonstrated that the HMB-FA supplementation group was significantly greater at 4, 8, and 12 weeks during the periodized resistance-training phases compared to the mean total strength in the placebo-supplemented group.

Wingate PP increased more in the HMB-FA-supplemented participants than in the placebo-supplemented participants over the 12-week study. HMB-FA-supplemented participants increased Wingate PP by 18% compared with a 12% increase in placebo-supplemented participants. The 12-week mean Wingate PP was also significantly greater in the HMB-FA group compared to the placebo group. A similar difference in vertical jump power was seen between the HMB-FA group and placebo group). Vertical jump power in the HMB-FA group increased 19% after 12 weeks compared to the placebo group increase of 12%. The mean vertical jump power was significantly improved with HMB-FA supplementation in comparison to the placebo at weeks 4, 8, and 12.

Comparison of the Effects of CaHMB and HMB-Acid on Strength and/or Function

FIG. 12 depicts the net percent increase in squat and bench press for CaHMB and HMB-acid based on the two studies described in this Example. The net percent increase per week in these strength or function parameters is almost 80% greater in individuals supplemented with HMB-acid compared to CaHMB. HMB-acid has improved efficacy over CaHMB for increasing strength gains by almost 80%. This is a significant and surprising result, demonstrating that the higher clearance rate of HMB-acid and longer time of exposure of tissues to HMB results in higher efficiency in this delivery form. The known effects of HMB (in this case improved muscle function as demonstrated by increases in muscle strength) are greater with the HMB-acid delivery form of HMB as compared to CaHMB. HMB-acid has improved efficacy over CaHMB for the known uses of HMB, including but not limited to improving muscle function and/or muscle strength.

The present invention demonstrates that HMB-acid is more readily available and has a higher clearance rate than CaHMB, resulting in higher efficiency and faster utilization. HMB-acid reaches a higher HMB plasma peak in a much shorter time than the CaHMB form, with similar urinary excretion of HMB. The combination of significantly higher peak plasma concentrations and AUC, coupled with no differences in urinary excretion over the measurement period indicates greater retention of HMB. The shorter half-lives and higher clearance rates from the plasma compartment are indicative of the increased utilization of the HMB free acid form. Administration of HMB-acid achieves higher sustained HMB plasma concentration that improves HMB bioavailability and efficacy to tissues. HMB-acid is more rapidly absorbed than CaHMB, resulting in a higher peak and sustained concentration in blood for maximum efficacy and results. HMB-acid increases clearance compared to CaHMB, resulting in greater retention and more HMB available to muscle tissue. HMB-acid has improved efficacy over CaHMB for improving the known effects of HMB, including increasing muscle mass and/or muscle function and/or muscle performance, including muscle strength and decreasing muscle degradation.

The foregoing description and drawings comprise illustrative embodiments of the present invention. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto. Those skilled in the art who have the disclosure before them will be able to make modifications and variations therein without departing from the scope of the invention. Administration of the composition of the present invention will be in an amount sufficient to achieve a desired effect as recognized by one of ordinary skill in the art.

### REFERENCE LIST

1. The Body Fluid Compartments: Extracellular and Intracellular Fluids; Interstitial Fluid and Edema. In: Textbook of Medical Physiology, edited by Guyton A C and Hall J E. Philadelphia: W B Saunders Company, 2007.
2. Adamson L F and Greenberg D M. The significance of certain carboxylic acids as intermediates in the biosynthesis of cholesterol. Biochim Biophys Acta 23: 472-479, 1957.
3. Bachhawat B K, Robinson W G and Coon M J. The enzymatic cleavage of beta-hydroxy-beta-methylglutaryl coenzyme a to aceto-acetate and acetyl coenzyme A. J Biol Chem 216: 727-736, 1955.
4. Bloch K, Clark L C and Haray I. Utilization of branched chain acids in cholesterol synthesis. J Biol Chem 211: 687-699, 1954.
5. Clark R H, Feleke G, Din M, Yasmin T, Singh G, Khan F and Rathmacher J A. Nutritional treatment for acquired immunodeficiency virus-associated wasting using β-hydroxy-β-methylbutyrate, glutamine and arginine: A randomized, double-blind, placebo-controlled study. JPEN J Parenter Enteral Nutr 24(3): 133-139, 2000.
6. Coon M J. Enzymatic synthesis of branched chain acids from amino acids. Fed Proc 14: 762-764, 1955.
7. Dreyer H C, Drummond M J, Pennings B, Fujita S, Glynn E L, Chinkes D L, Dhanani S, Volpi E and Rasmussen B B. Leucine-enriched essential amino acid and carbohydrate ingestion following resistance exercise enhances mTOR signaling and protein synthesis in human muscle. Am J Physiol Endocrinol Metab 294: E392-E400, 2008.
8. Dreyer H C, Fujita S, Cadenas J G, Chinkes D L, Volpi E and Rasmussen B B. Resistance exercise increases AMPK activity and reduces 4E-BP1 phosphorylation and protein synthesis in human skeletal muscle. J Physiol 576: 613-624, 2006.
9. Eley H L, Russell S T, Baxter J H, Mukherji P and Tisdale M J. Signaling pathways initiated by β-hydroxy-β-methylbutyrate to attenuate the depression of protein synthesis in skeletal muscle in response to cachectic stimuli. Am J Physiol Endocrinol Metab 293: E923-E931, 2007.
10. Eley H L, Russell S T and Tisdale M J. Attenuation of depression of muscle protein synthesis induced by lipopolysaccharide, tumor necrosis factor and angiotensin II by β-hydroxy-β-methylbutyrate. Am J Physiol Endocrinol Metab 295: E1409-E1416, 2008.
11. Eley H L, Russell S T and Tisdale M J. Mechanism of Attenuation of Muscle Protein Degradation Induced by Tumor Necrosis Factor Alpha and Angiotensin II by beta-Hydroxy-beta-methylbutyrate. Am J Physiol Endocrinol Metab 295: E1417-E1426, 2008.
12. Eubanks May P, Barber A, Hourihane A, D'Olimpio J T and Abumrad N N. Reversal of cancer-related wasting using oral supplementation with a combination of β-hydroxy-β-methylbutyrate, arginine, and glutamine. Am J Surg 183: 471-479, 2002.
13. Fuller, J. C., Jr., Sharp, R. L., Angus, H. F., Baier, S. M., and Rathmacher, J. A. Free acid gel form of β-hydroxy-β-methylbutyrate (HMB) improves HMB clearance from plasma in humans compared to the calcium HMB salt. Br. J. Nutr. 2010. Ref Type: In Press
14. Gallagher P M, Carrithers J A, Godard M P, Schulze K E and Trappe S W. β-Hydroxy-β-methylbutyrate ingestion, Part I: Effects on strength and fat free mass. Med Sci Sports Exerc 32(12): 2109-2115, 2000.
15. Gallagher P M, Carrithers J A, Godard M P, Schutze K E and Trappe S W. β-Hydroxy-β-methylbutyrate ingestion, Part II: Effects on hematology, hepatic, and renal function. Med Sci Sports Exerc 32(12): 2116-2119, 2000.
16. Gey K F, Pletsher A, Isler O, Ruegg R and Wursch J. Influence of iosoprenoid C5 and C6 compounds on the incorporation of acetate in cholesterol. Helvetica Chin Acta 40: 2354-2368, 1957.
17. Gey, K. F., Pletsher, A., Isler, O., Ruegg, R., and Wursch, J. The influence of isoperenic C5 and C6 compounds upon the acetate incorporation into cholesterol. Helvetica Chim. Acta 40, 2369. 1957. Ref Type: Abstract
18. Harper A E, Benevenga N J and Wohlhueter R M. Effects of ingestion of disproportionate amounts of amino acids. Physiol Rev 53: 428-558, 1970.
19. Hill, D. S., Szewczyk, N., Brookfield, R., Loughna, P., Rathmacher, J., Rennie, M. J., and Atherton, P. J. β-hydroxy-β-methylbutyrate (HMB) stimulates mammalian target of rapamycin complex 1 (mTORc1) signaling via a phosphatidylinositol-3-kinase (PI3K)-dependent pathway. FASEB J. 2011. Ref Type: Abstract
20. Isler, O., Ruegg, R., Wursch, J., Gey, K. F., and Pletsher, A. Biosynthesis of cholesterol from β,τ-dihydroxy-β-methylvaleric acid. Helvetica Chim. Acta 40, 2369. 1957. Ref Type: Abstract
21. Janssen G M E, Kuipers H, Willems G M, Does R J M M, Janssen M P E and Geurten P. Plasma activity of muscle enzymes: quantification of skeletal muscle damage and relationship with metabolic variables. Int J Sport 10: S160-S168, 1989.
22. Jowko E, Ostaszewski P, Jank M, Sacharuk J, Zieniewicz A, Wilczak J and Nissen S. Creatine and β-hydroxy-β-methylbutyrate (HMB) additively increases lean body mass and muscle strength during a weight training program. Nutr 17: 558-566, 2001.
23. Knitter A E, Panton L, Rathmacher J A, Petersen A and Sharp R. Effects of β-hydroxy-β-methylbutyrate on muscle damage following a prolonged run. J Appl Physiol 89(4): 1340-1344, 2000.
24. Krebs H A and Lund P. Aspects of the regulation of the metabolism of branched-chain amino acids. Advan Enzyme Regul 15: 375-394, 1977.
25. Nissen S, Panton L, Sharp R L, Vukovich M, Trappe S W and Fuller J C, Jr. β-Hydroxy-β-methylbutyrate (HMB) supplementation in humans is safe and may decrease cardiovascular risk factors. J Nutr 130: 1937-1945, 2000.
26. Nissen S, Sharp R, Ray M, Rathmacher J A, Rice J, Fuller J C, Jr., Connelly A S and Abumrad N N. Effect of the leucine metabolite β-hydroxy β-methylbutyrate on muscle metabolism during resistance-exercise training. J Appl Physiol 81(5): 2095-2104, 1996.
27. Nissen S, Van Koevering M and Webb D. Analysis of β-Hydroxy-β-methyl Butyrate in Plasma by Gas Chromatography and Mass Spectrometry. Anal Biochem 188(1): 17-19, 1990.
28. Nissen S, Van Koevering M and Webb D. Analysis of β-hydroxy-β-methyl butyrate in plasma by gas chromatography and mass spectrometry. Anal Biochem 188: 17-19, 1990.
29. Nissen S L and Abumrad N N. Nutritional role of the leucine metabolite β-hydroxy-β-methylbutyrate (HMB). J Nutr Biochem 8: 300-311, 1997.
30. Nissen S L and Sharp R L. Effect of dietary supplements on lean mass and strength gains with resistance exercise: a meta-analysis. J Appl Physiol 94: 651-659, 2003.
31. Nuviala R J, Roda L, Lapieza M G, Boned B and Giner A. Serum enzymes activities at rest and after a marathon race. J Sports Med Phys Fitness 32: 180-186, 1992.
32. Ostaszewski P, Kostiuk S, Balasinska B, Jank M, Papet I and Glomot F. The leucine metabolite 3-hydroxy-3-methylbutyrate (HMB) modifies protein turnover in muscles of the laboratory rats and domestic chicken in vitro. J Anim Physiol Anim Nutr (Swiss) 84: 1-8, 2000.
33. Panton L B, Rathmacher J A, Baier S and Nissen S. Nutritional supplementation of the leucine metabolite β-hydroxy β-methylbutyrate (HMB) during resistance training. Nutr 16(9): 734-739, 2000.
34. Phillips S M, Tipton K D, Aarsland A, Wolf S E and Wolfe R R. Mixed muscle protein synthesis and breakdown after resistance exercise in humans. Am J Physiol 273: E99-107, 1997.
35. Plaut G W E and Lardy H A. Enzymatic incorporation of C14-bicarbonate into acetoacetate in the presence of various substrates. J Biol Chem 192: 435-445, 1951.
36. Rabinowitz J L, Dituri F, Cobey F and Gurin S. Branched chain acids in the biosynthesis of squalene and cholesterol. Fed Proc 14: 760-761, 1955.
37. Rathmacher J A, Link G A, Flakoll P J and Nissen S L. Gas chromatographic-mass spectrometric analysis of stable isotopes of 3-methylhistidine in biological fluids: application to plasma kinetics in vivo. Biol Mass Spectrom 21: 560-566, 1992.
38. Rathmacher J A, Nissen S, Panton L, Clark R H, Eubanks M P, Barber A E, D'Olimpio J and Abumrad N N. Supplementation with a combination of beta-hydroxy-beta-methylbutyrate (HMB), arginine, and glutamine is safe and could improve hematological parameters. JPEN J Parenter Enteral Nutr 28: 65-75, 2004.
39. Rathmacher J A and Nissen S L. Development and application of a compartmental model of 3-methylhistidine metabolism in humans and domestic animals. Adv Exp Med Biol 445: 303-324, 1998.
40. Rennie M J. Exercise- and nutrient-controlled mechanisms involved in maintenance of the musculoskeletal mass. Biochem Soc Trans 35: 1302-1305, 2007.
41. Robinson W G, Bachhawat B K and Coon M J. Enzymatic carbon dioxide fixation by senecioyl coenzyme A. Fed Proc 13: 281, 1954.
42. Rudney H. The synthesis of β-hydroxy-β-methylglutaric acid in rat liver homogenates. J Am Chem Soc 76: 2595, 1954.
43. Rudney H and Farkas T G. Biosynthesis of branched chain acids. Fed Proc September: 757-759, 1955.
44. Russell S T and Tisdale M J. Mechanism of attenuation by beta-hydroxy-beta-methylbutyrate of muscle protein degradation induced by lipopolysaccharide. Mol Cell Biochem 330(1-2): 171-179, 2009.
45. SAS Institute Inc. SAS User's Guide: Statistics. Cary, N C: SAS Institute Inc., 1985.
46. Slater G, Jenkins D, Logan P, Lee H, Vukovich M D, Rathmacher J A and Hahn A G. b-hydroxy b-methylbutyrate (HMB) supplementation does not affect changes in strength or body composition during resistance training in trained men. Int J Sport Nutr Exerc Metab 11: 384-396, 2001.
47. Smith H J, Mukerji P and Tisdale M J. Attenuation of proteasome-induced proteolysis in skeletal muscle by β-hydroxy-β-methylbutyrate in cancer-induced muscle loss. Cancer Res 65: 277-283, 2005.
48. Smith H J, Wyke S M and Tisdale M J. Mechanism of the attenuation of proteolysis-inducing factor stimulated protein degradation in muscle by beta-hydroxy-beta-methylbutyrate. Cancer Res 64: 8731-8735, 2004.
49. Sousa M F, Abumrad N N, Martins C, Nissen S and Riella M C. Calcium β-hydroxy-β-methylbutyrate. Potential role as a phosphate binder in uremia: In vitro study. Nephron 72: 391-394, 1996.
50. Thalhammer F, Schenk P, Burgmann H, El M, I, Hollenstein U M, Rosenkranz A R, Sunder-Plassmann G, Breyer S and Ratheiser K. Single-dose pharmacokinetics of meropenem during continuous venovenous hemofiltration. Antimicrob Agents Chemother 42: 2417-2420, 1998.
51. Urso R, Blardi P and Giorgi G. A short introduction to pharmacokinetics. Eur Rev Med Pharmacol Sci 6: 33-44, 2002.
52. Vukovich M D, Slater G, Macchi M B, turner M J, Fallon K, Boston T and Rathmacher J. β-Hydroxy-β-methylbutyrate (HMB) kinetics and the influence of glucose ingestion in humans. J Nutr Biochem 12: 631-639, 2001.
53. Vukovich M D, Stubbs N B and Bohlken R M. Body composition in 70-year old adults responds to dietary β-hydroxy-β-methylbutyrate (HMB) similar to that of young adults. J Nutr 131(7): 2049-2052, 2001.
54. Zabin I and Bloch K. The utilization of butyric acid for the synthesis of cholesterol and fatty acids. J Biol Chem 192: 261-266, 1951.

1. A method of improving muscle utilization of β-Hydroxy-β-methylbutyrate (HMB) comprising administering to a human a composition comprising HMB in the free acid form (HMB-acid), wherein the administration of HMB-acid results in an improvement in at least one measure of muscle utilization of HMB, said measures comprising improving muscle function, improving muscle performance, improving muscle strength, compared to administration of an equivalent amount of HMB in the calcium salt form (CaHMB).

2. The method of claim 1, wherein the measure of muscle utilization of HMB is increased muscle strength.

3. The method of claim 2, wherein muscle strength is increased by at least ten percent compared to administration of an equivalent amount of CaHMB.

4. The method of claim 1, wherein the composition is provided in a delivery form selected from the list consisting of a capsule, a liquid or a gel.

5. The method of claim 1, wherein the composition comprises 0.5 g to 30 g of HMB-acid.

6. A method of increasing muscle strength in a human comprising administering to a human an effective amount of a composition comprising β-Hydroxy-β-methylbutyrate (HMB) in the free acid form (HMB-acid), wherein the administration of HMB-acid results in greater increases in muscle strength than administration of an equivalent amount of HMB in the calcium salt form (CaHMB).

7. The composition of claim 6, wherein the composition is provided in a delivery form selected from the list consisting of a capsule, a liquid or a gel.

8. The composition of claim 6, wherein the composition comprises 0.5 g to 30 g of HMB-acid.

9. A method of increasing tissue bioavailability of β-Hydroxy-β-methylbutyrate (HMB) in a human comprising administering to said human a composition comprising HMB in the free acid form (HMB-acid), wherein said HMB-acid exhibits increased bioavailability in comparison to an equivalent amount of HMB administered in the calcium salt (CaHMB) form.

10. The method of claim 9, wherein the increased bioavailability results in greater improvements in muscle strength, muscle performance, and/or muscle function as compared to administration of an equivalent dose of CaHMB.

11. The method of claim 9, wherein the composition is provided in a delivery form selected from the list consisting of a capsule, a liquid or a gel.

12. The method of claim 9, wherein the composition comprises 0.5 g to 30 g of HMB-acid.

13. A method of optimizing the effects of HMB on muscle tissue comprising administering a composition to a human of β-Hydroxy-β-methylbutyrate (HMB) in the free acid form (HMB-acid), wherein administration of HMB-acid results in at least one physiological improvement in the human selected from the list of increasing muscle strength, improving in muscle function, improving in muscle performance and increasing muscle mass as compared to administration of a similar amount of HMB in the calcium salt form (CaHMB).

14. The method of claim 13, wherein the HMB-acid is provided with a delivery form selected from the list consisting of a capsule, a liquid or a gel.

15. The method of claim 13, wherein the HMB-acid is provided in a delivery form selected from the list consisting of a capsule, a liquid or a gel.

16. A finished food-grade composition comprising β-Hydroxy-β-methylbutyrate (HMB) in the free acid form (HMB-acid), wherein said composition when orally administered to a human provides greater improvements in at least one of the physiological effects of HMB compared to oral administration of an equivalent dose of HMB in the calcium salt form (CaHMB).

17. The composition of claim 16, wherein the physiological effects are selected from the list consisting of increasing muscle strength, improving in muscle function, improving in muscle performance and increasing muscle mass.

18. The composition of claim 16, wherein the composition is provided in a delivery form selected from the list consisting of a capsule, a liquid or a gel.

19. The composition of claim 16, wherein the composition comprises 0.5 g to 30 g of HMB-acid.
